(19) United States
(12) Patent Application Publication
(10) Pub. No.: US 2009/0170066 A1
(43) Pub. Date: Jul. 2, 2009

(54) METHODS AND MATERIALS THEREFOR

(75) Inventors: Brigitte Desiree Alberte Colau, Rixensart (BE); Evert Wilhelmus Hoefnagel, Voorburg (NL); Gijsbertus Everardus Kleter, Voorburg (NL); Annick Poliszczak, Rixensart (BE); Wilhelmus Gregorius Quint, Voorburg (NL); Leendert Jan Van Doorn, Voorburg (NL)

Correspondence Address: GLAXOSMITHKLINE;Corporate Intellectual Property - UW2220, P.O. Box 1539, King of Prussia,, PA, 19406-0939, US

(73) Assignee: GLAXOSMITHKLINE BIOLOGICALS S.A.

(21) Appl. No.: 11/909,008

(22) PCT Filed: Mar. 16, 2006
(86) PCT No.: PCT/EP06/02542

§ 371 Date: Sep. 18, 2007

(30) Foreign Application Priority Data
Mar. 18, 2005 (GB) 0505620.5

(51) Int. Cl. C12Q 1/70; C07H 21/04

(52) U.S. Cl. 435/5; 536/24.33; 536/24.32

(57) ABSTRACT

The present invention relates to a method for detecting or detecting and identifying rotavirus in a biological sample. In particular, the invention relates to a detection method comprising contacting the nucleic acids from the sample or derived from the sample with at least one VP4 and/or VP7 universal probes in the context of a solid support and detecting any type-specific hybridisation. The invention further relates to a detection or detection followed by typing method comprising contacting the nucleic acids from the sample or derived from the sample with at least one P type-specific and G type-specific probes in the context of a solid support and detecting any type-specific hybridisation. The invention also relates to primers and probes used therein and to diagnostic kits.

### FIELD OF THE INVENTION

The present invention relates to a method for detecting or detecting and identifying rotavirus in a biological sample. In particular, the invention relates to a detection method comprising contacting the nucleic acids from the sample or derived from the sample with at least one VP4 and/or VP7 universal probes in the context of a solid support and detecting any type-specific hybridisation. The invention further relates to a detection or detection followed by typing method comprising contacting the nucleic acids from the sample or derived from the sample with at least one P type-specific and G type-specific probes in the context of a solid support and detecting any type-specific hybridisation. The invention also relates to primers and probes used therein and to diagnostic kits.

### BACKGROUND OF THE INVENTION

Acute, infectious diarrhoea is a leading cause of disease and death in many areas of the world. In developing countries, the impact of diarrhoeal disease is staggering. Each year and worldwide, rotavirus causes 111 million episodes of infantile gastroenteritis requiring only home care, 25 million cases requiring a visit to a clinic, 2 million hospitalisations and 352,000-592,000 deaths in children <5 years of age (Parashar U D et al.: Emerg Infect Dis 2003; 9(5):565-72).

Rotaviruses have been recognised as one of the most important causes of severe diarrhoea in infants and young children (Estes, M. K. Rotaviruses and Their Replication in Fields Virology, Third Edition, edited by Fields et al., Raven Publishers, Philadelphia, 1996). It is estimated that rotavirus disease is responsible for over one million deaths annually. Rotavirus-induced illness most commonly affects children between 6 and 24 months of age, and the peak prevalence of the disease generally occurs during the cooler months in temperate climates, and year-round in tropical areas. Rotaviruses are typically transmitted from person to person by the faecal-oral route with an incubation period of from about 1 to about 3 days. Unlike infection in the 6-month to 24-month age group, neonates are generally asymptomatic or have only mild disease. In contrast to the severe disease normally encountered in young children, most adults are protected as a result of previous rotavirus infection so most adult infections are mild or asymptomatic (Offit, P. A. et al. Comp. Ther., 8(8):21-26, 1982).

Rotaviruses, classified as a genus in the Reoviridae family, are generally spherical and their name is derived from their distinctive outer and inner or double-shelled capsid structure. Typically, the double-shelled capsid structure of a rotavirus surrounds an inner protein shell or core that contains the genome. The genome of a rotavirus is composed of 11 segments of double-stranded RNA which encode six structural and six non-structural viral proteins (Fischer T K & Gentsch J R, 2004, Rev. Med. Virol. 14, 71-82). Two of these viral proteins designated as VP4 and VP7 are arranged on the exterior of the double-shelled capsid structure. VP4 protein is a spike protein and is the translational product of genomic segment 4, whilst VP7 belongs to the outer capsid and is the translational product of genomic segment 7, 8 or 9, depending on the strain. The inner capsid of the rotavirus presents one protein, which is the rotavirus protein designated VP6. The relative importance of these three particular rotaviral proteins in eliciting the immune response that follows rotavirus infection is not yet clear. Nevertheless, the VP6 protein determines the group and subgroup antigen. More specifically, antigenicity is based on five VP6 groups (A, B, C, D, and E) and four subgroups within VP6 group A (I, II, I+II, neither I nor II) (Fischer T K & Gentsch J R, see above). Furthermore, another specificity is based on VP4 protein and VP7 protein which are the determinants of serotype specificity. VP4, designated as P, is a non-glycosylated protease-sensitive protein of approximately 88 kD. This protein stimulates neutralising antibody following rotavirus infection. VP7 protein, a glycoprotein of 38 kD (34 kD when non-glycosylated) and designated as G, stimulates formation of the major neutralising antibody following rotavirus infection. One of the nonstructural proteins of relevance, NSP4, is a transmembrane, endoplasmic reticulum-specific glycoprotein with pleiotropic functions in viral replication and pathogenesis. NSP4, encoded by gene segment 10 of group A rotavirus, is an enterotoxin causing diarrhea and is described as an activator of a signal transduction pathway that increases intracellular calcium levels in cells by mobilising calcium from the endoplasmic reticulum, resulting in chloride secretion (Zhang et al. J. of Virology, 72, 3666-3672, 1998).

To date, 10 G serotypes and 10 P serotypes have been detected by serological assays in human rotaviruses, although five G (G1, G2, G3, G4 and G9) and two P (P8 and P4) are most commonly found. In general, strains sharing more than 89% amino acid identity are considered to belong to the same P genotype. The nomenclature for G genotypes and serotypes is identical (followed by an open number), whereas a P genotype is denoted by closed brackets and serotype is indicated by an open number (O'Mahoney, J. et al., JCM 1999, 37; 6:1699-1703).

Early vaccine development for preventing rotavirus infections began in the 1970s after the discovery of the virus. Several vaccine approaches exist based upon homotypic or heterotypic protection provided against either a single common G serotype (monovalent vaccines) or against multiple serotypes (multivalent vaccines). These approaches comprise live attenuated strains from animals, live attenuated strains from humans such as that described in U.S. Pat. No. 5,474,773 (deposited strain 89-12C2) (see also Bernstein, D. L. et al, Vaccine, 16 (4), 381-387, 1998) or that described in WO 01/12797 which is a monovalent vaccine based on an attenuated human rotavirus strain 89-12 belonging to the G1P[8] genotype (deposited strain ECACC 99081301), or human-animal reassortants.

Rotavirus strain surveillance is of high importance in disease control programmes worldwide, to ensure that i) epidemiology and new strains prevalence is monitored, ii) subsequent to the introduction of a vaccine, the circulating strains continue to be monitored, iii) reassortment of animal rotavirus genes from the field into human strains are monitored, and iv) the rotavirus vaccine composition can be updated to include the most common G and P serotypes (Fischer T K & Gentsch J R, see above).

Detection and characterisation of rotavirus strains present in biological samples such as in foodstuffs, water or faecal extracts for example, have been reported in the literature. Several methods have been described amongst which immunoassays such as enzyme immunoassays with polyclonal or monoclonal antibodies or passive particle agglutination tests, or molecular approaches based on the amplification of the viral RNA such as the reverse transcription-polymerase chain reaction (RT-PCR) technique and the nucleic acid sequence-based amplification (NASBA) followed by either gel electrophoresis or microtiter plate hybridization of the amplified products. The amplification-based typing techniques have proven to be of great value in typing the rotavirus-positive specimens untypable by the serologic technique. Different approaches have been used to detect and type rotavirus RNA:
Use of a broad-spectrum PCR primer set designed in a conserved region to amplify VP4 or VP7 rotavirus RNA followed by Southern blot (or slot blot) hybridizations with different digoxigenin-labelled G and P type-specific oligonucleotide probes sequentially (Leite J P et al 1996, Ramachandran M et al 1996)
Use of a broad spectrum PCR primer set combined with type-specific PCR primers set to selectively amplify VP4 (P2, 4, 6, 8) or VP7 (G1-4, G8-9) and gel electrophoresis analysis to match the size of the PCR product with reference DNA products (Gouvea et al 1990 and Das et al 1994).
Use of a broad spectrum PCR primer set to amplify VP7 (G1-4, G9) Rotavirus RNA, followed by a second round of nested PCR to generate a fluorescent labelled sample and by an hybridization step with oligonucleotide microarrays (Chizhikov V et al, 2002).

These detection/typing methods however still suffer from several disadvantages because they request two consecutive PCR's which increases the potential problem of carry-over contamination, gel electrophoresis analysis with or without different hybridizations which are labor intensive step. None of them are really suitable for high throughput analysis.

There is a need therefore for alternative rotavirus typing methods, in particular for VP4, VP7, VP6 and/or NSP4-typing method of rotaviruses, in particular but not only human rotavirus, which are more sensitive and/or specific or easier to carry out and more reliable compared to conventional typing methods by PCR and which are appropriate for validation and automation.

Multiparameter testing vs. single testing, broad-spectrum amplification vs genotype specific, easier discrimination, detection of multiple infections. (mixed genotypes), higher throughput than e.g. gel. Completeness of the algorithm. Robustness, specificity, reproducibility, etc.

We now describe a novel rotavirus typing method, which overcome the drawbacks of the prior art methods.

### DESCRIPTION OF FIGURES

FIG. 1—Human rotavirus outer capsid protein (VP4) nucleic acid sequence of P8 Wa-strain (GenBank accession number L34161)

FIG. 2—Human rotavirus gene 9 encoding viral glycoprotein VP7 nucleic acid sequence of G1 Wa-strain (GenBank accession number M21843)

FIG. 3—Schematic representation of the Reverse hybridisation line probe assay

FIG. 4—Methodology/algorithm to detect and/or genotype rotavirus in a biological sample

FIG. 5—Illustration of LiPA strips for the different genotypes. FIG. 5A illustrates a VP7 strip, FIG. 5B illustrates a VP4 strip

FIG. 6—Outline of the VP4 rotavirus Reverse hybridisation line probe assay for detection and identification of P-genotypes

FIG. 7—Outline of the VP7 rotavirus Reverse hybridisation line probe assay for detection and identification of G-genotypes

FIG. 8—Amplified VP7 gene fragments following amplification using specific primer sets (A, B, C and D described in Table 12)

FIG. 9—Human rotavirus outer capsid protein (VP4) nucleic acid sequence of G1P[8] strain deposited under ECACC 99081301 (WO 01/12797)

FIG. 10—Human rotavirus gene 9 encoding viral glycoprotein VP7 nucleic acid sequence of G1P[8] strain deposited under ECACC 99081301 (WO 01/12797)

### STATEMENT OF THE INVENTION

The present invention relates to a method for detecting or detecting and typing rotavirus, in particular, human rotavirus, in a biological sample, e.g. for diagnosis and identification of sequence heterogeneity in the rotaviral genome, associated with rotavirus-induced infections. In particular, the invention relates to a detection method comprising contacting the nucleic acids from the sample or derived from the sample with at least one VP4 Universal probe (‘Uniprobe’) and/or at least one VP7 Uniprobe in the context of a solid support; and detecting a rotavirus P- or G-type hybridisation. The invention further relates to a detection/typing method comprising the steps of (i) contacting the nucleic acids from the sample or derived from the sample with at least one, preferably a plurality of G type-specific and at least one P type-specific probe(s) in the context of a solid support; and (ii) detecting any type-specific hybridisation.

In a further aspect the method according to the invention comprises a preliminary step of amplifying rotavirus nucleic acids from the sample by use of broad-spectrum rotavirus primers and subsequent use of said amplified nucleic acids in step (i).

In a further aspect the method comprises an additional step whereby the nucleic acid sequences detected with the G type-specific or P type-specific probes are analysed with at least one, preferably a plurality of, VP4 and/or VP7 universal probes (Uniprobes) to confirm the presence of rotavirus nucleic acid simultaneously or sequentially with the type-specific hybridisation step (ii).

In another aspect, the invention relates to primers and probes suitable for use in said rotavirus detection and/or amplifying method. In a still further aspect of the invention, diagnostic kits for use in the rotavirus detection and/or typing and method are provided.

### DETAILED DESCRIPTION OF THE INVENTION

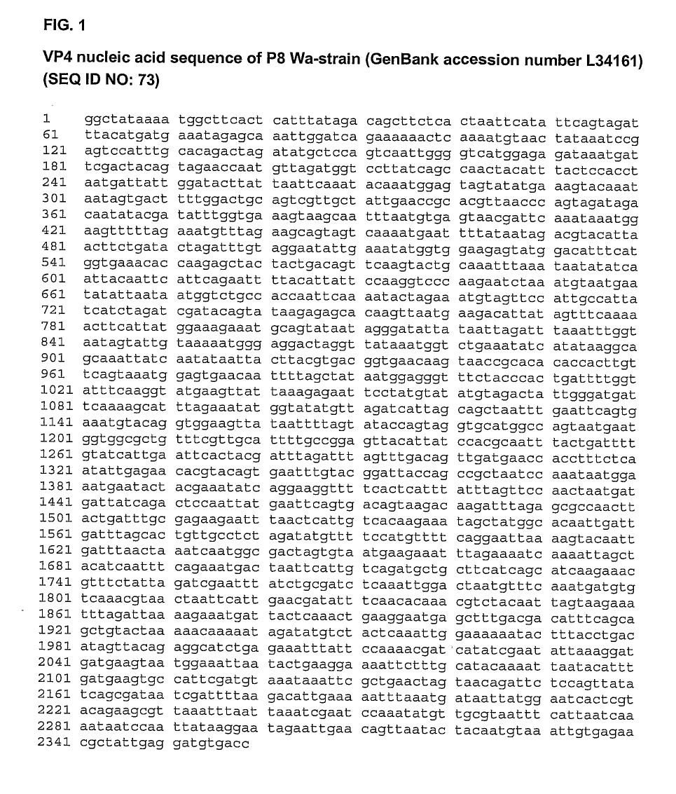

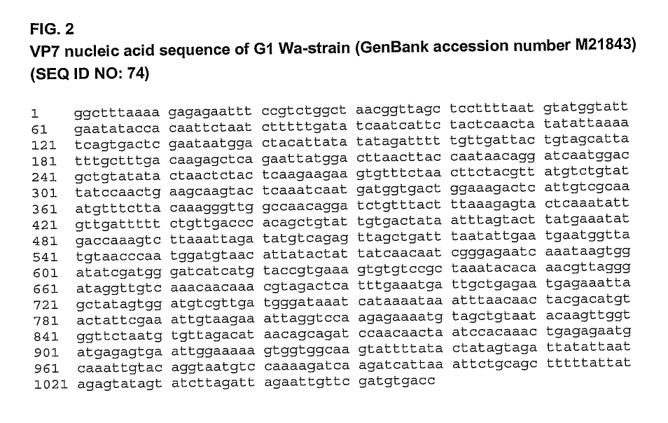

The present invention provides a method for detecting the presence of a rotaviral nucleic acid possibly present in a biological sample, comprising the steps of:
(i) contacting any such nucleic acid from the sample or derived from the sample with at least one VP4 Uniprobe and/or at least one VP7 Uniprobe in the context of a solid support; and
(ii) detecting hybridization of said uniprobe to any such nucleic acid in the sample, or derived from the sample;
   wherein said VP4 probe is capable of hybridisation within the nucleic acid region of nucleotides 640-685 of VP4, said VP4 sequence being set forth in FIG. 1, or within an equivalent region in another rotavirus VP4 sequence, and
   wherein said VP7 probe is capable of hybridisation within the nucleic acid region of nucleotides 852-878 of VP7, said VP7 sequence being set forth in FIG. 2, or within an equivalent region in another rotavirus VP7 sequence.

Such VP4 Uniprobes assess the presence of rotavirus VP4 nucleic acid sequences, i.e. the presence of P-type rotavirus sequences. Similarly, for the G-typing, the VP7 Uniprobes assess the presence of rotavirus VP7 nucleic acid sequences.

### Universal Probes or ‘Uniprobes’

It can be useful to check for the presence of rotavirus RNA prior to any specific typing analysis. In this case the use of universal probes which are able to recognize any rotavirus sequence may be employed. Where amplification of target is carried out prior to specific typing then universal probes are located within the amplified region. Examples of universal probes that may be used to verify the presence of rotavirus RNA are listed in Table 1 or 2.

Universal probes may contain inosine residues as part of the nucleic acid probe sequence, which allows for some flexibility in hybridisation to target nucleic acid, and can allow hybridisation to different rotavirus type-specific sequences.

Universal probes may be used to detect rotavirus nucleic acid using the DEIA technique, for example as explained in WO 99/1437 and for example in Clin Diagn Virol. 1995 February; 3(2):155-64, herein incorporated by reference. This method is used for rapid and specific detection of PCR products. PCR products are generated by a primer set, of which either the forward or the reverse primer contains biotin at the 5′ end. This allows binding of the biotinylated amplimers to streptavidin-coated microtiter wells. PCR products are denatured by sodium hydroxide, which allows removal of the non-biotinylated strand. Specific labelled oligonucleotide probes (e.g. with digoxigenin) are hybridized to the single stranded immobilized PCR product and hybrids are detected by enzyme-labelled conjugate and colorimetric methods.

Suitable VP4 and VP7 Uniprobes are indicated in Table 1 and 2, respectively. For example, a VP4 uniprobe is that which is capable of hybridisation to the 640-685 nucleic acid region of VP4, said VP4 reference sequence being set forth in FIG. 1, or within an equivalent region in another rotavirus VP4 sequence. For example, a VP7 uniprobe is that which is capable of hybridisation to the 852-878 nucleic acid region of VP7, said VP7 reference sequence being set forth in FIG. 2, or within an equivalent region in another rotavirus VP7 sequence.

Suitable VP4 Uniprobes are selected from the sequences set forth in Table 1 (SEQ ID NO: 1 to 6). Suitable VP7 Uniprobes are selected from the sequences set forth in Table 2 (SEQ ID NO: 20 to 24).

TABLE 1

Universal Probes for identification of rotavirus VP4
types (‘VP4 Uniprobes’)
VP4 Uniprobes are referred to as VP4-uni_1, VP4-uni_2,
VP4-uni_3, VP4-uni_4, VP4-uni_5 and VP4-uni_6.

| Probe | | |
|---|---|---|
| designation | Sequence 5′→3′ (SEQ ID NO) | Position¹ |
| VP4-uni_1 | TAAACCATTATTAATATATTCATTACANTTAGANTCTT | 678-640 |
| | G | |
| | (SEQ ID NO: 1) | |
| VP4-uni_2 | ACCGTTGTTAATATATTCATTACANTTAGANTCTTG | 675-640 |
| | (SEQ ID NO: 2) | |
| VP4-uni_3 | TTGGTGGNAANCCAGTATTTATATATTCA | 685-657 |
| | (SEQ ID NO: 3) | |
| VP4-uni_4 | TTGGTGGTAACCCNTTATTTATGTANTCAG | 685-656 |
| | (SEQ ID NO: 4) | |
| VP4-uni_5 | GGTGGTAAACCATTATTTATATATTGTGTACACATAG | 683-647 |
| | (SEQ ID NO: 5) | |
| VP4-uni_6 | TTGGAGGCANCCCATTATTTATATATTGCGC | 685-655 |
| | (SEQ ID NO: 6) | |

¹All sequence positions according to sequence of the P8 Wa-strain (GenBank accession number L34161).
N stands for Inosine.

TABLE 2

Universal Probes for identification of rota-
virus VP7 types (‘VP7 Uniprobes’)
VP7 Uniprobes are referred to as Rota-uni_2,
Rota-uni_3, Rota-uni_4, Rota-uni_5 and Rota-
uni_6.

| Probe | | |
|---|---|---|
| designation | Sequence 5′→3′ | Position¹ |
| Rota-uni_2 | ATTAGACATAACAGCAGATCCAACGAC | 852-878 |
| | (SEQ ID NO: 20) | |
| Rota-uni_3 | TAGATATCACTGCTGATCCAACAAC | 854-878 |
| | (SEQ ID NO: 21) | |
| Rota-uni_4 | ATTAGATATAACGGCTGATCCCACAAC | 852-878 |
| | (SEQ ID NO: 22) | |
| Rota-uni_5 | TAGATATTACAGCTGATCCGACGAC | 854-878 |
| | (SEQ ID NO: 23) | |
| Rota-uni_6 | TTGGATATTACNGCNGATCCAACAAC | 853-878 |
| | (SEQ ID NO: 24) | |

¹All sequence positions according to sequence of the G1 Wa-strain (GenBank accession number M21843).
N stands for Inosine.

In a specific embodiment, more than one, i.e. a plurality of VP4 and/or VP7 uniprobes is used on the solid support. A plurality of universal probes is intended to mean two or more VP4 or two or more VP7 probes. In particular, two or more, i.e. 3, 4, 5, 6 or more VP4 Uniprobes are used, and are for example selected from the sequences set forth in Table 1 (SEQ ID NO: 1 to 6).

In another embodiment, two or more, i.e. 3, 4, 5 or more VP7 Uniprobes are used, and are for example selected from the sequences set forth in Table 2 (SEQ ID NO: 20 to 24). For example, the VP4 Uniprobe(s) are selected from the sequences set forth in Table 1 (SEQ ID NO: 1 to 6). Similarly, the VP7 Uniprobe(s) are for example selected from the sequences set forth in Table 2 (SEQ ID NO: 20 to 24). In particular, the detection method comprises the use of all 5 VP4 Uniprobes as set forth in Table 1 and/or all 6 VP7 Uniprobes as set forth in Table 2. Any combination of VP4 and VP7 probes is also contemplated in the present invention. For example, all 5 VP4 Uniprobes and all 6 VP7 Uniprobes as depicted in Tables 1 and 2 respectively can be present on the solid support. The VP4 and VP7 probes may be associated on the same solid support, e.g. on the same strip membrane of e.g. nitrocellulose or nylon, or may be found on distinct solid supports such as solid beads e.g polystyrene solid beads of the commercially available Luminex™ technology.

The present invention further provides a method for detecting and/or typing a rotavirus, in particular human rotavirus, possibly present in a biological sample, comprising the steps of:
(i) contacting the nucleic acids of any such rotavirus from the sample or derived from the sample with at least one VP4 type-specific probe in the context of a solid support; and
(ii) detecting type-specific hybridisation so obtained;
   wherein the VP4 type-specific probe is capable of hybridising to the nucleic acid region of nucleotides 204-703 of VP4, said VP4 sequence being set forth in FIG. 1, or to an equivalent region in another rotavirus VP4 sequence.

Specifically said VP4 type-specific probe is capable of hybridising to a sequence anywhere within the interprimer nucleic acid region. Suitably the VP4 type-specific probe is capable of hybridising to the nucleic acid region between the most 5′ and the most 3′ probe in the selection, and suitably to nucleic acid region of nucleotides 234-537 of VP4, said VP4 sequence being set forth in FIG. 1, or to an equivalent region in another rotavirus VP4 sequence.

In a specific embodiment, said method claimed herein above additionally comprises the steps of:
(i) contacting the nucleic acids from the sample or derived from the sample with at least one VP7 type-specific probe in the context of a solid support; and
(ii) detecting type-specific hybridisation;
   wherein the VP7 type-specific probe is capable of hybridising to the nucleic acid region of nucleotides 529-928 of VP7, said VP7 sequence being set forth in FIG. 2, or to an equivalent region in another rotavirus VP7 sequence.

Specifically said VP7 type-specific probe is capable of hybridising to a sequence anywhere within the interprimer nucleic acid region. Suitably the VP7 type-specific probe is capable of hybridising to the nucleic acid region between the most 5′ and the most 3′ probe in the selection, and suitably to the nucleic acid region of nucleotides 602-840 of VP7, said VP7 sequence being set forth in FIG. 2, or to an equivalent region in another rotavirus VP7 sequence.

In another embodiment, the present invention further provides a method for detecting and/or typing a rotavirus, in particular human rotavirus, possibly present in a biological sample, comprising the steps of:
(i) contacting the nucleic acids of any such rotavirus from the sample or derived from the sample with at least one VP7 type-specific probe in the context of a solid support; and
(ii) detecting type-specific hybridisation;
   wherein the VP7 type-specific probe is capable of hybridising to the nucleic acid region of nucleotides 529-928, suitably to the nucleic acid region of nucleotides 602-840 of VP7, said VP7 sequence being set forth in FIG. 2, or to an equivalent region in another rotavirus VP7 sequence.

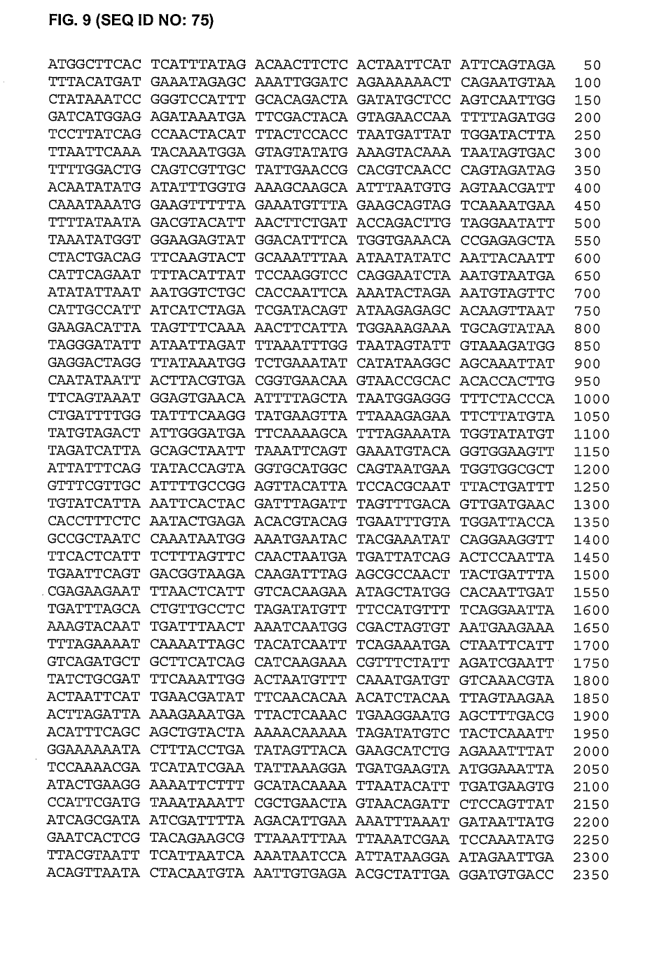

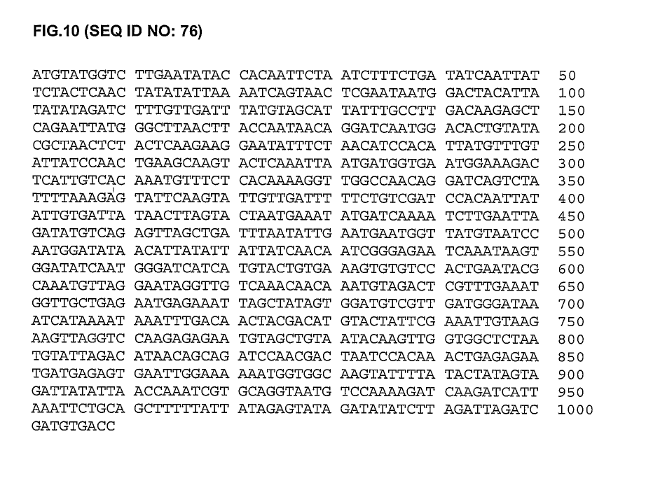

In particular, type-specific sequences will be selected so as to be exclusive to a specific rotavirus strain, preferably of human origin. By way of example, the attenuated human G1P[8] rotavirus vaccine strain deposited under ECACC 99081301 has a specific signature compared to the wild type strain (WO 01/12797, incorporated by reference). At least one mutation is present in either the VP7 or VP4 wild-type gene sequence, compared to the sequence in the wild-type rotavirus strain. Specifically, in VP4 gene sequence, a A is present in the wild-type strain at position 501 of VP4 nucleotide sequence (amino acid residue 167) as set forth in FIG. 9, whilst a T is found in the corresponding VP4 sequence of the deposited vaccine strain, resulting into an amino acid change (from leucine to phenylalanine). In addition, A nucleotide is found at positions 788 (amino acid 263) and 802 (amino acid 268) of the VP4 vaccine strain sequence (instead of a G in the wild-type sequence), resulting into an amino acid change from glycine to either glutamic acid (position 263) or arginine (position 268). Similarly, at least one VP7 gene sequence mutation from C to T is found at position 605 (corresponding to amino acid 202) of VP7 sequence as set forth in FIG. 10. This translates to an amino acid mutation from threonine to methionine.

In the context of the present invention, the terms VP4 type-specific probe or P-type specific probe are interchangeably used. Similarly, the terms VP7 type-specific probe or G-type specific probe are interchangeably used.

By ‘VP4 or VP7 type-specific probe’ is meant a probe which will hybridize to such a part of the amplified VP4 or VP7 region, which is indicative of the presence of a distinct genotype. Such a type-specific probe will only hybridize to this distinct genotype, while not hybridizing (or to a much less effectiveness) to a sequence of another distinct genotype. In some cases, genotypes can be determined by a combination of type-specific probes.

By ‘equivalent region’ is meant equivalent regions of other rotavirus VP4 or VP7 sequences (which may vary from the sequence used as a reference), where the equivalent region is identified on the basis of, for example, sequence homology or identity with the sequence of FIG. 1 or of FIG. 2.

Sequence comparisons of nucleic acid identity/homology are readily carried out by the skilled person, for example using the BLAST and BLAST 2.0 algorithms, which are described in Altschul et al., Nucl. Acids Res. 25:3389-3402 (1977), and Altschul et al., J. Mol. Biol. 215:403-410 (1990), respectively. BLAST and BLAST 2.0 can be used, for example with the default parameters, to determine percent sequence identity for the polynucleotides of the invention. Software for performing BLAST analyses is publicly available through the National Center for Biotechnology Information.

In a particular aspect of the invention, said P-type specific probes are specific for at least one of: P1, P3, P4, P6, P8_{WT}, P8_{vac}, P9 and P10, and said G-type specific probes are specific for at least one of G1_{WT}, G1_{vac}, G2, G3, G4, G5, G6, G8, G9 and G12. For example, the probes for identification of P-genotypes are selected from the list set forth in Table 3 (SEQ ID NO:7 to 19 and 66), and the probes for identification of G-genotypes are selected from the list set forth in Table 4 (SEQ ID NO:25 to 45 and SEQ ID NO: 67 to 72).

Accordingly it is an aspect of the present invention to provide for a method for detecting and/or typing a rotavirus, in particular human rotavirus, in a biological sample, comprising the steps of:
(i) contacting the nucleic acids from the sample or derived from the sample with at least one, preferably a plurality of, G type-specific and at least one, preferably a plurality of, G type-specific or P type-specific probes in the context of a solid support; and
(ii) detecting any type-specific hybridisation;
   wherein the G type-specific probes are specific for at least one of G1_{WT}, G1_{vac}, G2, G3, G4, G5, G6, G8, G9 and G12, and the P type-specific probes are specific for at least one of: P1, P3, P4, P6, P8_{WT}, P8_{vac}, P9 and P10, and
   wherein the probes for identification of P-genotypes are selected from the list set forth in Table 3 (SEQ ID NO:7 to 19 and 66), and wherein the probes for identification of G-genotypes are selected from the list set forth in Table 4 (SEQ ID NO:25 to 45 and SEQ ID NO: 67 to 72).

TABLE 3

Probes for identification of specific
P-genotypes

| Probe | Sequence 5′→3′ | |
|---|---|---|
| designation | (SEQ ID NO) | Position¹ |
| P1_3_1 | CAAATAGGTGGTTAGCGA | 305-322 |
| | (SEQ ID NO: 7) | |
| P1_3_2 | CAAACAGATGGTTAGCGA | 305-322 |
| | (SEQ ID NO: 8) | |
| P4_1_1c | AATAGACTTGTAGGAATGCTAA | 490-511 |
| | (SEQ ID NO: 9) | |
| P4_2c | TTCGAAATGTTTAAAGGTAGC | 427-447 |
| | (SEQ ID NO: 10) | |
| P6_3 | TATAATAGTGTTTGGACTTTC | 517-537 |
| | (SEQ ID NO: 11) | |
| P8_VAC-str1b | CTGATACCAGACTTGTAGGA | 485-504 |
| | (SEQ ID NO: 12) | |
| P8_VAC-str2b | GTAGGAATATTTAAATATGGTG | 499-520 |
| | (SEQ ID NO: 13) | |
| P8_1_2b | ACCACCTACTGATTACTGG | 234-252 |
| | (SEQ ID NO: 14) | |
| P8_4_1a | ACCCAGTAGATAGACAATATATG | 347-369 |
| | (SEQ ID NO: 15) | |
| P8_4_3a | GATCCAGTAGATAGACAATATAATG | 346-370 |
| | (SEQ ID NO: 16) | |
| P9_1c | ACTTCGTGGAAATTTATATTATT | 415-437 |
| | (SEQ ID NO: 17) | |
| P_9_2d | AGATGGGCAAAATGTCCAAGG | 372-392 |
| | (SEQ ID NO: 18) | |
| P10b | AGCCCCATTAAATGCTG | 261-277 |
| | (SEQ ID NO: 19) | |
| P8_WT probe | AGTGCAGTGGATAGACAATATACTG | 347-369 |
| | (SEQ ID NO: 66) | |

¹All sequence positions according to sequence of the G1 Wa-strain (GenBank accession number M21843).
N stands for Inosine.

TABLE 4

Probes for identification of specific
G-genotypes

| Probe | | |
|---|---|---|
| designation | Sequence 5′→3′ | Position¹ |
| G1vac-605T1 | GAATACGCAAATGTTAGG | 642-659 |
| | (SEQ ID NO: 25) | |
| G1WT-605C3 | AATACGCAAACGTTAGGA | 643-661 |
| | (SEQ ID NO: 26) | |
| G1WT-605aC4 | ACTGAATACACAAACGTTAG | 639-658 |
| | (SEQ ID NO: 27) | |
| G1WT-605taC1 | CTGAATATACAAACGTTAGG | 640-659 |
| | (SEQ ID NO: 28) | |
| G1WT-1_2 | TTAGCTATAGTGGATGTCGGG | 718-739 |
| | (SEQ ID NO: 29) | |
| G1WT-1a_10 | AAATTAGCTATAGTAGATGTCGGG | 715-739 |
| | (SEQ ID NO: 30) | |
| G1WT-2_3 | CGTTGATGGGATAAATCAT | 735-753 |
| | (SEQ ID NO: 31) | |
| G1WT-2t_10 | CGTTGATGGGATAAATTAT | 735-753 |
| | (SEQ ID NO: 32) | |
| G2_11 | AGAAAATGTTGCTATAATTCA | 813-833 |
| | (SEQ ID NO: 33) | |
| G3_11 | AGCAGTTATACAGGTTGGT | 822-840 |
| | (SEQ ID NO: 34) | |
| G3B_1 | GGCGGTCATACAAGTT | 822-837 |
| | (SEQ ID NO: 35) | |
| G4_11 | CAGCTACTTTTGAAACAGTT | 683-702 |
| | (SEQ ID NO: 36) | |
| G4VA70-3 | AGCTACTTTTGAAATGGTG | 684-702 |
| | (SEQ ID NO: 37) | |
| G4_Arg1 | CAGCTACTTTTGAAACGGGG | 683-702 |
| | (SEQ ID NO: 38) | |
| G5_2 | TATCTATGGGTTCTTCATGG | 602-624 |
| | (SEQ ID NO: 39) | |
| G6_2 | CTCGGTATCGGATGTCT | 655-671 |
| | (SEQ ID NO: 40) | |
| G8_n10 | ACTACAACTTTTGAAGAAGTTGC | 682-704 |
| | (SEQ ID NO: 41) | |
| G8_n21 | ACACTACGACTTTTGAAGAA | 680-699 |
| | (SEQ ID NO: 42) | |
| G9_1 | ATGGGACAGTCTTGTAC | 607-623 |
| | (SEQ ID NO: 43) | |
| G12_1 | GAAGAGGTAGCAAACG | 694-709 |
| | (SEQ ID NO: 44) | |
| G12_4 | AGGTAGCAAACGCGG | |
| | (SEQ ID NO: 45) | 697-711 |
| G2 NEW1 | AGAGAATGTTGCTATAATTCA | 813-833 |
| | (SEQ ID NO: 67) | |
| G3 NEW1 | AGCAGTTATACAGGTAGGT | 822-840 |
| | (SEQ ID NO: 68) | |
| G4 NEW1 | CAGTTACTTTTGAAACAGTT | 683-702 |
| | (SEQ ID NO: 69) | |
| G8 NEW1 | ACGACCACTTTCGAGGAAGTTGC | 682-704 |
| | (SEQ ID NO: 70) | |
| G9_NEW1 | ATGGGACAATCTTGTACC | 607-624 |
| | (SEQ ID NO: 71) | |
| G9 NEW2 | ATAGGACAATCTTGTACC | 607-624 |
| | (SEQ ID NO: 72) | |

¹All sequence positions according to sequence of the G1 Wa-strain (GenBank accession number M21843).
N stands for Inosine.

According to one embodiment, a plurality of G-type specific and P-type specific probes are present on the solid support. A plurality of type-specific probes is intended to mean two or more G-type specific and/or two or more P-type specific probes, i.e. 3, 4, 5, 6, 7, 8, 9, 10 or more G-type specific probes and/or P-type specific probes, or any combination thereof. For example, 11 P-type probes and 19 G-types probes as depicted in Tables 3 and 4 can be present on the solid support.

The G-type and P-type probes may be combined on the same solid support, e.g. on the same strip membrane of nitrocellulose or nylon, or may be separate on distinct solid supports such as solid beads e.g polystyrene solid beads of the commercially available Luminex™ technology. For example, the P-type probes, both universal and specific, may be combined on the same strip, and the G-type probes, both universal and specific, may be combined on another strip. Alternatively, strips for P and G types (both universal and specific) may be combined.

Optionally, said methods as claimed herein may further comprise the step of confirming the rotavirus G- and/or P-type by sequencing.

The instant invention further provides for a detection/typing method combining the use of VP4 and/or VP7 Universal rotavirus probes that are designed to recognize a broad range of rotavirus nucleic acid and types together with P-specific and/or G-specific type probes that are designed for actual genotyping of the sequences. Therefore, in another specific embodiment, said nucleic acid sequences detected with the type specific probes are analysed with at least one of VP4 and/or VP7 Uniprobes as herein defined, to confirm the presence of rotavirus nucleic acid. This additional step may be performed either sequentially or simultaneously with the hybridisation step involving the type specific sequences.

Accordingly, in another aspect there is provided a method for detecting and/or typing a rotavirus in a biological sample, comprising the steps of:
(i) contacting the nucleic acids from the sample or derived from the sample with at least one VP4 type-specific probe(s) and at least one VP7 type-specific probe(s) in the context of a solid support; and
(ii) detecting any type-specific hybridisation;
   wherein the nucleic acid sequences are analysed with at least one of VP4 and/or VP7 Uniprobes to confirm the presence of rotavirus nucleic acid sequentially (i.e. prior to) or simultaneously with the hybridisation step (ii); and
   wherein the probes (type-specific and universal) are as hereinbefore described.

The assay is performed on a solid support, for example a microarray or a strip. Suitably when detection is combined with genotyping the assay is carried out on a strip. Alternatively a microarray or any other suitable solid support may be used, such as solid beads e.g polystyrene solid beads of the commercially available Luminex™ technology.

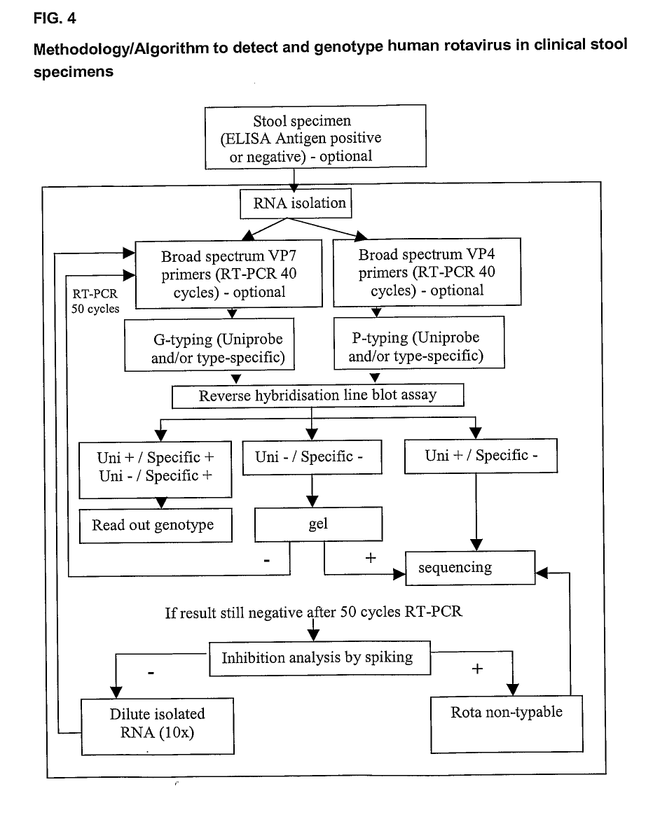

The methodology/algorithm of the reverse hybridisation (e.g. in line) assay is schematically represented in FIG. 4.

Universal probes may also allow detecting variant rotavirus sequences, though they do not allow for a specific typing of the newly detected sequences. In other words, universal probes will allow recognising i.e. hybridising to variant types of rotavirus sequences without enough specificity to allow typing of the detected sequences. In particular, Universal probes may also allow detecting new rotavirus sequences.

As said above, to determine the genotype of a rotavirus present in a clinical isolate, VP4 and/or VP7 sequences can be used. Sequences can be amplified by reverse transcriptase PCR (RT-PCR) using broad-spectrum primers, aimed at conserved parts of the genomic region of interest, e.g., the primers described in this application. The amplified products can be sequenced by standard methods (Sanger dideoxy sequencing). The resulting sequence should be classified by comparison to known sequences, which have been assigned to a specific genotype. This can be achieved by performing multisequence alignments using specific software packages, such as Vector NTI, Clustal. etc. (there is a variety of programs available). During this alignment process, the differences between any pair of sequences (molecular distance) is calculated and several algorithms are available, such as the Jukes and Cantor parameters. The calculated molecular distances can be graphically represented in a phylogenetic tree.

Thus, based on the molecular distance to any of the known rotavirus reference sequences, and based on the position in the phylogenetic tree, a novel sequence can be classified (Molecular evolution and phylogenetics. M. Nei and S. Kumar. Oxford University Press, New York, 2000. ISBN 0-19-513584-9; Fundamentals of molecular evolution. D. Graur and W-H Li. Sinauer Associates Inc., Sunderland, USA, 2000. ISBN 0-87893-266-6). If the novel sequence shows a high homology (which equals a low molecular distance) to known reference sequence, the novel sequence has the same genotype as the reference sequence. In contrast, if the novel sequence shows a low homology (which equals a high molecular distance), to any of the known reference sequences, the novel sequence should be classified as a novel genotype. However, to define a novel genotype, it is advisable to analyse different regions of the viral genome.

It can be envisaged that a novel rotavirus genotype will show a positive signal to any of the universal probes, present on the hybridization support, but that it will result in an aberrant hybridization pattern on the genotype-specific probes, or will not hybridize to any of the genotype-specific probes. This is what is meant by a variant rotavirus sequence.

Accordingly in another aspect of the invention it is provided for a process for detecting the presence of rotavirus sequence variants in a biological sample comprising the steps of:

i) contacting a VP7 and/or VP4 Uniprobe with the nucleic acids from the sample
ii) detecting hybridization of a rotavirus G or P type sequences,
iii) contacting the nucleic acid in the sample with the P and/or G type-specific probe
wherein said Uniprobe and said type-specific probes are as herein defined, and
(iv) identify said P— pr G-type.

In another optional aspect of the invention, said detection and/or typing methods additionally comprise a first step of amplifying rotavirus nucleic acids from the sample by use of broad-spectrum rotavirus primers and subsequent use of said amplified nucleic acids in step (i) of any of the methods hereinbefore described. This additional step proves advantageous to detect the pathogen's presence. This additional step may alternatively prove useful to increase the amount of starting material (i.e. low concentrations of rotavirus nucleic acids) from the sample under testing and facilitate the subsequent detection/typing steps.

Accordingly, in another embodiment, the method additionally comprises a further step wherein the nucleic acids amplified by use of broad spectrum primers in the first optional step are analysed to confirm the presence of polynucleotide types of interest prior to the hybridization step (i). Only those samples positive for the general nucleic acids types of interest may then be screened in step (ii) of the detection and detection/typing methods as herein described.

In particular, the broad-spectrum rotavirus primers are designed to amplify VP4 and/or VP7 RNA from the sample. For example, the amplimers resulting from the first preliminary amplification step are then contacted with VP4 and/or VP7 Universal rotavirus probes that are designed to recognize a broad range of rotavirus nucleic acid and types and are as defined hereabove. This general detection step can be performed before the actual P-specific and G-specific genotyping step or may be performed simultaneously with the genotyping step. It is carried out on a solid support. Suitably the assay is carried out in a microtitre plate, which allows analysing the presence/absence of multiple sequences, as described in Kleter et al., 1998, American J. of Pathology 153, 1731-1739. Alternatively, a strip or any other suitable solid support such as beads may be used.

In an aspect of the invention, the broad-spectrum rotavirus primers are designed to amplify VP4 and/or VP7 RNA from the sample.

In a specific embodiment, the VP4 broad-spectrum primers comprise at least one forward (5′) primer hybridising to the 204-224 nucleic acid region of VP4, and at least one reverse (3′) primer hybridising to the 680-703 nucleic acid region of VP4, said VP4 sequence being set forth in FIG. 1, or to an equivalent region in another rotavirus VP4 sequence. When alternatively counted from the ATG the at least one forward (5′) primer hybridises to the 195-216 nucleic acid region of VP4, said VP4 sequence being set forth in FIG. 1. Similarly, when alternatively counted from the ATG the at least one reverse (3′) primer hybridises to the 671-695 nucleic acid region of VP4, said VP4 sequence being set forth in FIG. 1.

In another specific embodiment, the VP7 broad-spectrum rotavirus primers comprise at least one forward (5′) primer hybridising to the 529-553 nucleic acid region of VP7, and at least one reverse (3′) primer hybridising to the 907-928 nucleic acid region of VP7, said VP7 sequence being set forth in FIG. 2, or to an equivalent region in another rotavirus VP7 sequence.

For example, the VP4 broad-spectrum primers are selected from the sequences set forth in Table 5 (SEQ ID NO: 46 to 54) and the VP7 broad-spectrum primers are selected from the sequences set forth in Table 6 (SEQ ID NO: 55 to 65).

In one specific embodiment the VP4 broad-spectrum primers are a cocktail of two or more forward and reverse primers, for example a cocktail of all six forward and three reverse primers as set forth in Table 5. In another specific embodiment, the VP7 primers are a cocktail of two or more forward and reverse primers, for example a cocktail of all six forward and five reverse primers as set forth in Table 6.

TABLE 5

Primers used for amplification of the VP4 gene
fragment of human rotavirus

| Primer | | |
|---|---|---|
| designation | Sequence 5′→3′ | position¹ |
| ROT4FOR1A | AGATGGTCCTTATCAGCCAAC | 204-224 |
| | (SEQ ID NO: 46) | |
| ROT4FOR1B | GANGGTCCTTATCAGCCTAC | 205-224 |
| | (SEQ ID NO: 47) | |
| ROT4FOR1C | AGATGGTCCTTATCAACCTAC | 204-224 |
| | (SEQ ID NO: 48) | |
| ROT4FOR1D | CGATGGTCCTTATCAACCAAC | 204-224 |
| | (SEQ ID NO: 49) | |
| ROT4FOR1E | GACGGACCATATCAACCGAC | 205-224 |
| | (SEQ ID NO: 50) | |
| ROT4FOR1F | GATGGTCCNTATCAACCNAC | 205-224 |
| | (SEQ ID NO: 51) | |
| ROT4REV1B | TTNCTTGTATTCTGNATTGGTG | 701-680 |
| | (SEQ ID NO: 52) | |
| ROT4REV1A | CATTTCTAGTATTTTGAATTGGTG | 703-680 |
| | (SEQ ID NO: 53) | |
| ROT4REV1C | CATTTCTAGTGTTTTGTATCGGT | 703-681 |
| | (SEQ ID NO: 54) | |

¹All sequence positions according to sequence of the P8 Wa-strain (GenBank accession number L34161).
N stands for Inosine.
Forward primers (FP): SEQ ID NO: 46 through to 51; Reverse primers (RP): SEQ ID NO: 52 through to 54.
Amplimer size = 500 bp

TABLE 6

Primers used for amplification of the VP7 gene
fragment of human rotavirus

| Primer | | |
|---|---|---|
| designation | Sequence 5′→3′ | position¹ |
| ROTFOR1A | AATGAATGGTTATGTAATCCAATGG | 529-553 |
| | (SEQ ID NO: 55) | |
| ROTFOR1B | AANGAATGGCTGTGCAATCCNATGG | 529-553 |
| | (SEQ ID NO: 56) | |
| ROTFOR1C | AANGAGTGGTTATGTAATCCNATGG | 529-553 |
| | (SEQ ID NO: 57) | |
| ROTFOR1D | AATGAATGGTTATGNAACCCAATGG | 529-553 |
| | (SEQ ID NO: 58) | |
| ROTFOR1E | AATGAGTGGCTTTGTAATCCAATGG | 529-553 |
| | (SEQ ID NO: 59) | |
| ROTFOR1F | AATCAATGGTTATGTAATCCGATGG | 529-553 |
| | (SEQ ID NO: 60) | |
| VP7rev4a | GCCACCATTTTTTCCAATTCAC | 928-907 |
| | (SEQ ID NO: 61) | |
| VP7rev4b | GCCACCATTTTTTCCAATTTAT | 928-907 |
| | (SEQ ID NO: 62) | |
| VP7rev4c | GCCACCATTTTTTCCAATTTAC | 928-907 |
| | (SEQ ID NO: 63) | |
| VP7rev4d | GCCACCATTTCTTCCAATTAAC | 928-907 |
| | (SEQ ID NO: 64) | |
| VP7rev4e | GCCACCATTTTTTCCAGTTCAC | 928-907 |
| | (SEQ ID NO: 65) | |

¹All sequence positions according to sequence of the G1 Wa-strain (GenBank accession number M21843).
N stands for Inosine.
Forward primers (FP): SEQ ID NO: 55 through to 60; Reverse primers (RP): SEQ ID NO: 61 through to 65.
Amplimer size = 400 bp

In another aspect of the present invention, the rotavirus nucleic acid sequences are further analysed with rotavirus probes other than G- or P— probes, both type-specific and/or universal probes. As a non-limiting example, said probes will be capable of hybridisation with other rotavirus genes, such as VP6 or NSP4 for example or with a combination of both.

The present invention provides that, in the aforementioned methods of detection and/or typing of rotavirus, the hybridisation step involves a reverse hybridisation format, in particular on a solid support. Such a format implies that the probes are immobilised to specific locations on said solid support and that the rotavirus nucleic acids, whether amplified or not, are hybridized to the probe and detected. According to one embodiment, at least one probe is used. According to another embodiment, a plurality of probes are used, for example such as a set of at least 2, or 3, or 4 or 5 or 6 or 7 or 8 or 9 or 10 or more probes is used. When at least a set of 2 or more probes is used, the skilled man will understand that the length and the design of the probe sequences may have to be adapted in order to be able to conduct the hybridisation at one and the same hybridization condition (e.g. ionic strength and temperature) for all the probes.

The term ‘solid support’ as used herein can refer to any substrate to which a nucleic acid probe can be coupled, provided that it retains its hybridisation characteristics and provided that the background level of hybridisation remains low. For example the solid support may be a membrane (e.g. made of nylon or nitrocellulose), a microtiter plate or a microsphere (bead) or a microarray.

According to a particular embodiment, the aforementioned hybridisation step is a reverse hybridization assay, such as for example a reverse line probe technique. Such assay involves a reverse hybridisation step, characterised in that the oligonucleotide probes are immobilised on a solid support (e.g. a strip of nitrocellulose or nylon) as parallel lines as described in WO 94/12670, in WO 99/14377, and in Kleter et al, [Journal of Clinical Microbiology (1999), 37(8):2508-2517], the whole contents of which are herein incorporated by reference.

Such an assay offers several advantages as compared to other hybridisation formats, especially when a combination of several probes is needed to obtain the information sought. Such technique is especially suitable when large numbers of samples are to be tested, and is also appropriate for validation purposes (e.g. robustness, specificity, and reproducibility).

Moreover, this format allows highly specific hybridisation analysis under stringent conditions, permitting single mismatches between probe and target sequence to be detected. This assay is used to detect the cDNA of rotavirus and to identify/type several P genotypes and several G genotypes. In one specific embodiment, at least one G-specific probe and one P-specific probe are fixed onto the strip for further hybridisation with the rotavirus nucleic acids from the sample. For example 2 or more, e.g. 3, 4, 5, 6, 7, 8, 9, 10, 11, 12, 13, 14, 15, 16, 17, 18, 19, 20 or more of each G- and/or P-genotype-specific probes, selected from the group consisting of: G1_{WT}, G1_{vac}, G2, G3, G4, G5, G6, G8, G9, G12, P1, P3, P4, P6, P8_{WT}, P8_{vac}, P9 and P10, are fixed onto the strip.

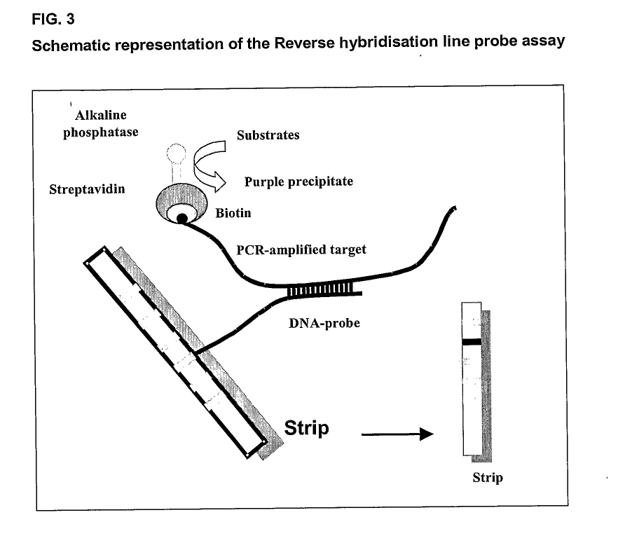

Typically the type-specific probes are hybridised with the biotinylated amplicons, in particular with the generic PCR fragments of rotavirus gene, such as the 400 bp long amplified fragment for the VP7 gene, and 500 bp long amplified fragment for the VP4 gene. The hybrids are recognised by the alkaline phosphatase-streptavidin conjugate and detected by colour developing in the presence of alkaline phosphatase substrates, where the conjugate appears as an insoluble and purple precipitating product resulting in visible colouring of a probe line. A schematic representation of the Reverse hybridisation line probe assay is shown in FIG. 3. Alternatively the type-specific probes can directly, i.e. without prior amplification by PCR, be hybridised with the rotavirus nucleic acid sequences (i.e. rotavirus RNA genome) within the sample. In this example, the hybrids are recognized by a biotinylated oligonucleotide probe (such as the PCR primer used when the rotavirus material is amplified) and followed by the alkaline phosphatase-streptavidin conjugate detection step.

The probes of the invention are about 5 to 50 nucleotides long, more preferably from about 10 to 25 nucleotides. Particularly suitable probe lengths are optimised under given circumstances and include 10, 11, 12, 13, 14, 15, 16, 17, 18, 19, 20, 21, 22, 23, 24, or 25 nucleotides. The nucleotides to be used in the present invention may be ribonucleotides, deoxyribonucleotides and modified nucleotides such as inosine or nucleotides containing modified groups which do not negatively impact on their hybridisation properties.

In a particular embodiment, the reverse hybridisation (e.g. in line) assay includes a combination of at least one universal probe (i.e. a VP4 and/or VP7 Uniprobes), to confirm the presence (or amplification) of VP7 and/or VP4 sequences and at least one pair of G and P-type specific probes to identify the G- and P-genotype. An example of a suitable VP7 reverse hybridisation assay includes 5 Uniprobes and 19 G-specific probes (to identify G1_{WT}, G1_{vac}, G2, G3, G4, G5, G6, G8 and G9), as indicated in Table 4. It may additionally comprise G12-specific probes as indicated in Table 4. Similarly, a suitable VP4 reverse hybridisation assay includes 6 Uniprobes and 11 P-specific probes (to identify P4, P6, P8_{WT}, P8_{vac}, P9 and P10), as indicated in Table 3. As indicated in Table 3, P1 and/or P3-specific probes may additionally be present.

Other solid support techniques may be used according to the invention. When the solid support is a microtiter plate, the DNA Enzyme Immunoassay (DEIA) technique may be used to rapidly and specifically detect rotavirus nucleic acids and/or PCR products in a biological sample, such as described in Kleter et al., 1998, American J. of Pathology 153, 1731-1739.

It is also an aim of the invention to provide oligonucleotide sequences, e.g. primers and probes, enabling the hereinbefore-described methods of detection and/or amplification of rotavirus sequences.

The invention also relates to sets of oligonucleotides, said sets comprising at least one primer set and/or at least one probe that may be used to perform the methods of detection and/or identification of rotavirus sequences as described above.

Preferred primers and probes according to the present invention can for example be chosen from the sequences set forth in Tables 1 to 6.

Accordingly, a broad-spectrum primer for use in the amplification of rotavirus nucleic acid sequences in a biological sample or derived from a biological sample, may be selected from the list consisting of: a VP7 primer set or a VP4 primer set as defined above, or a combination thereof. Particularly contemplated broad-spectrum primer sequences are as set forth in Tables 5 and 6.

Specifically, the invention extends to cover any primer listed in Table 5 or 6, and pairs of primers useful in amplification of region 204-703, suitably in region 195-695 of rotavirus VP4 sequence (given by reference to FIG. 1) or in amplification of region 529-928 of rotavirus VP7 sequence (given by reference to FIG. 2), or to an equivalent region in another rotavirus VP4 or VP7 sequence, or to smaller fragments within said region.

A Uniprobe for use in the detection and/or identification of rotavirus nucleic acid sequence in a biological sample or derived from a biological sample, may be selected from the list consisting of: a VP4 or a VP7 Uniprobe as defined above, or a combination thereof. Particularly contemplated Uniprobe sequences are as set forth in Tables 1 and 2. Specifically, the invention extends to cover any Uniprobe listed in Table 1 or 2 which hybridises within the nucleic acid region of nucleotides 640-685 of VP4 (given by reference to FIG. 1), or within the nucleic acid region of nucleotides 852-878 of VP7 (given by reference to FIG. 2).

A rotavirus type-specific probe for use in typing rotavirus nucleic acid sequence in a biological sample or derived from a biological sample may be selected from the list consisting of: a VP4-type specific probe as defined above, a VP7-type specific probe as defined above, or a combination thereof. Particularly contemplated type-specific sequences are as set forth in Tables 3 and 4. Specifically, the invention extends to cover any type-specific probe listed in Table 3 or 4 which hybridises within the nucleic acid region of nucleotides 204-703, suitably in region of nucleotides 234-537 of VP4 (given by reference to FIG. 1), or within the nucleic acid region of nucleotides 529-928, suitably within the nucleic acid region of nucleotides 602-840 of VP7 (given by reference to FIG. 2).

The length and the design of the probe sequences can be optimised to be used in a given format, such as the reverse hybridisation line probe assay format for instance, under the same hybridisation and washing conditions. Evidently when other hybridisation conditions would be preferable, all probes will be readily adapted according to routine sequence optimisation protocols, for example by the addition or deletion of one or more nucleotides at their extremities. It is to be understood that these optimisations should not negatively impact on the hybridisation of the probes to their (type)-specific target sequences.

The invention also relates to the reverse complement of all type-specific probe sequences or Uniprobes sequences.

The present invention also provides for protocols and diagnostic kits according to which the hereinbefore-described hybridisation and amplification steps can be performed.

Accordingly there is provided a kit comprising at least two Uniprobes as defined herein above. In another embodiment there is provided a kit comprising at least two type-specific probes as defined herein above. In a specific embodiment said Uniprobes and type-specific probes are attached to a solid support.

In another embodiment the invention provides a kit comprising at least one primer as defined herein above and at least one Uniprobe as defined herein above, optionally with instructions for carrying out the above methods for rotavirus detection and/or typing.

In a specific embodiment the invention provides a kit comprising at least one set of VP4 Uniprobes and VP4-type specific probes as herein defined, or least one set of VP7 Uniprobes and/or VP7-type specific probes as herein defined or a combination of both, optionally with instructions for carrying out the above methods for rotavirus detection and/or typing. Said kit may further comprise a set of suitable broad spectrum primers as defined in the present invention.

Accordingly there is provided a diagnostic kit for detection and/or identification of rotavirus possibly present in a biological sample, comprising:

(i) at least one set of VP-7 Uniprobes and G-type specific probes as herein defined, fixed to a solid support, or
(ii) at least one set of VP-4 Uniprobes and P-type specific probes as herein defined, fixed to a solid support, or
(iii) a combination of both.

Typically a VP7 or VP4 diagnostic kit according to the present invention comprises the following components:

(i) at least one set of VP-7 Uniprobes and G-type specific probes as herein defined, fixed to a solid support;
(ii) at least one set of VP-4 Uniprobes and P-type specific probes as herein defined, fixed onto a solid support;
(ii) optionally at least one suitable primer pair;
(ii) a hybridisation buffer, or components to prepare said hybridisation buffer, or instructions to prepare said buffer;
(iv) a wash solution, or components to prepare said solution, or instructions to prepare said solution;
(v) optionally a means for detecting of the hybrids formed;
(vi) optionally a means for attaching the probe(s) to a known location on a solid support.

The term ‘hybridization buffer’ means a buffer allowing a hybridization reaction between the probes and the polynucleic acids present in the sample, or the amplified products, under the appropriate stringency conditions.

The term ‘wash solution’ means a solution enabling washing of the hybrids formed under the appropriate stringency conditions.

It is understood that a kit may further contain a protocol for carrying out the reaction, and standard reagents such as positive control, PCR reagents (e.g. buffer, MgCl2, nucleotides), and standard reaction buffers such as a denaturation buffer, a hybridization buffer, a stringent washing buffer, a rinse solution, etc. It may further contain a reading card for interpretation of results.

Suitably, the VP4/P and VP7/G detection/identification probes are onto the same solid support, in particular onto the same strip or within the same well on a microtiter plate. Alternatively the VP4/P and VP7/G detection/identification probes are onto distinct solid support. It is to be understood that this alternative will depend on the hybridisation conditions (nature of the probe, length, hybridisation temperature, etc) and on the number of probes to be used.

The following definitions will permit a better understanding of the present invention.

Rotavirus G- and P-types are defined in Mary K. Estes [2001 in Fields Virology vol 2 Fourth edition p 1747-chapter 54.

The method of the invention uses a ‘biological sample’. This term refers to any biological material, taken from an individual being tested for infection and/or risk of disease. Body solids such as stools or internal body tissues such as the intestine, or fluids such as urine, saliva and blood, may be used in the method of the invention. They may be taken directly from the body or may be enriched through culturing (cultured strains for example). Alternatively a ‘biological sample’ may also refer to biological/environmental material such as water or food.

A ‘probe’ according to the invention refers to a single-stranded nucleic acid sequence which is designed to hybridise specifically to rotavirus nucleic acid sequences. The probe may be labelled to permit specific detection of hybrids.

A ‘primer’ according to the invention refers to a single-stranded oligonucleotide sequence that is capable of acting as a point of initiation for synthesis of a primer extension product that is complementary to the nucleic acid strand to be copied. The design (length and specific sequence) of the primer will depend on the nature of the DNA and/or RNA targets and on the conditions at which the primer is used (such as temperature and ionic strength). A ‘primer pair’ refers to a pair of primers allowing for the amplification of part or all of the rotavirus polynucleotide fragment (the ‘amplimer’) for which probes are able to bind or for which the probes are immobilised on the solid support.

The ‘target sequence’ of a probe or a primer is a rotavirus nucleic acid sequence (either DNA or RNA, e.g. genomic DNA, messenger RNA, viral RNA or amplified versions thereof) to which the probe or primer is partially (i.e. with some degree of mismatch) or, preferably, totally complementary. These molecules are in this application also termed “nucleic acids” or “polynucleic acids. Well-known extraction and purification procedures are available for the isolation of RNA or DNA from a sample (e.g. in Sambrook et al., 1989). It may be used directly from the sample or, more preferably, after a polynucleotide amplification step (e.g. PCR) step. In specific instances such as for the reverse hybridisation assay, it may be necessary to reverse transcribe RNA into cDNA before amplification. In both latter cases the amplified polynucleotide is ‘derived’ from the sample. A ‘type-specific target sequence’ refers to a rotavirus target sequence that contains at least one nucleotide difference as compared to another rotavirus genotype/variant.

‘Specific hybridization’ of a probe to a region of the rotavirus polynucleic acids means that said probe forms a duplex with part of this region or with the entire region under the experimental conditions used, and that under those conditions said probe does not form a duplex with other regions of the polynucleic acids present in the sample to be analysed. It should be understood that probes that are designed for specific hybridisation within a region of rotavirus polynucleic acid may fall entirely within said region or may to a large extent overlap with said region (i.e. form a duplex with nucleotides outside as well as within said region). Suitably the specific hybridisation of a probe to a nucleic acid target region occurs under stringent hybridisation conditions, such as 3×SSC, 0.1% SDS, at 50° C.

The skilled person knows how to vary the parameters of temperature, probe length and salt concentration such that specific hybridisation can be achieved. Hybridization and wash conditions are well known and exemplified in Sambrook, et al., Molecular Cloning: A Laboratory Manual, Second Edition, Cold Spring Harbor, N.Y., (1989), particularly Chapter 11 therein. When needed, slight modifications of the probes in length or in sequence can be carried out to maintain the specificity and sensitivity required under the given circumstances. Probes and/or primers listed herein may be extended by 1, 2, 3, 4 or 5 nucleotides, for example, in either direction.

Preferred stringent conditions are suitably those which allow for a type specific probe binding to only one rotavirus type. Thus in an embodiment of the invention the method for typing of any rotavirus nucleic acid possibly present in a biological sample comprises the steps of contacting any such nucleic acid with at least one probe which is capable of hybridisation to the VP4 and/or VP7 sequence under stringent conditions.

Probes which specifically hybridise to the rotavirus genome as defined herein suitably at least 95% complementary to the target sequence over their length, suitably greater than 95% identical such as 96%, 97%, 98%, 99% and most preferably 100% complementary over their length to the target rotavirus sequence. The probes of the invention can be complementary to their target sequence at all nucleotide positions, with 1, 2, or more mismatches possibly tolerated depending upon the length of probe, temperature, reaction conditions and requirements of the assay, for example.

Suitably each nucleotide of the probe can form a hydrogen bond with its counterpart target nucleotide.

Preferably the complementarity of probe with target is assessed by the degree of A:T and C:G base pairing, such that an adenine nucleotide pairs with a thymine, and such that a guanine nucleotide pairs with a cytosine, or vice versa. In the RNA form, T may be replaced by U (uracil).

Where inosine is used in universal probes, for example, then hybrisisation to different rotavirus type-specific sequences is allowed at the mismatch position between the probe and the target.

Hybridisation of the polynucleotides may be carried out using any suitable hybridisation method and detection system. Examples of hybridisation systems include conventional dot blot, Southern blots, and sandwich method for example. For example it includes a reverse hybridisation approach, wherein type-specific probes are immobilised on a solid support in known distinct locations (dots, lines or other figures), and amplified polynucleic acids are labelled in order to detect hybrid formation.

In particular is the reverse hybridisation line probe system used as described above and in the Examples section, wherein the selected set of oligonucleotide probes are immobilised to a membrane strip in a parallel line fashion. The probes may be immobilised individually or as mixtures to delineated locations on the solid support. The rotavirus nucleic acid sequences can be labeled with biotin and the hybrid can be detected via a biotin-streptavidin coupling with a non-radioactive colour developing system. However, other reverse hybridisation systems may also be employed, for example, as illustrated in Gravitt et al, (Journal of Clinical Microbiology, 1998, 36(10): 3020-3027) the contents of which are also incorporated by reference. Standard hybridisation and wash conditions are described in Kleter et al., Journal of Clinical Microbiology, 1999, 37(8): 2508-2517 and will be optimised under the given circumstances to maintain the specificity and the sensitivity required by the length and sequence of the probe(s) and primer(s).

Throughout this specification and the claims which follow, unless the context requires otherwise, the word “comprise”, and variations such as “comprises” and “comprising”, will be understood to imply the inclusion of a stated integer or step or group of stated integers or steps but not to the exclusion of any other integer or step or group of integers or steps. ‘Comprising’ also implies the inclusion of the meanings, ‘consisting of’ and ‘consisting essentially of’.

The invention will be further described by reference to the following, non-limiting, examples:

### Example I

### Detection and Genotyping of Human Rotavirus in Clinical Stool Specimens: Outline of the Methodology

An algorithm has been developed which examplifies a possible strategy used for Human Rotavirus genotyping by the VP7 and VP4 RT-PCR Rota Reverse hybridisation line probe assay, and is illustrated in FIG. 4.

The algorithm is suitable for the testing of stool samples collected from clinical trials as large as Phase III clinical trials, and comprises the following steps:

### I.1. Nucleic Acid Extraction

Total nucleic acid is extracted from the stool samples (20% w/v in Earl's balanced salt solution (SIGMA® Ref E2888)) using the MagNAPure automated system and the MagNAPure LC DNA isolation kit III purchased from Roche, cat. number 3 264 785 and according to the manufacturer's instructions. Briefly, cells are first lysed by warming at 65° C. and addition of proteinase K lysis buffer to the sample, then incubated at 95° C. for 10 min to inactivate the proteinase K before being mixed with silica coated paramagnetic particles. These particles selectively bind nucleic acid. After separation of the paramagnetic particles from the extract, using a magnet, and several washing steps, RNA is eluted in 100 μl elution buffer (included in the Roche kit) from the paramagnetic particles.

### I.2. RT-PCR

I.2.1. A 400-bp RNA fragment is transcribed into cDNA which is amplified by using a cocktail of 11 primers (see Table 6), 6 forward and 5 reverse primers, to amplify at least 8 known HRV G-genotypes (G1_{WT}, G1_{vac}, G2, G3, G4, G5, G6, G8 and G9).

I.2.2. A 500-bp RNA fragment is transcribed into cDNA which is amplified by using a cocktail of 9 primers (see Table 4), 6 forward and 3 reverse primers, to effectively amplify at least 5 known P-genotypes (P4, P6, P8_{WT}, P8_{vac}, P9 and P10).

All reverse primers carry a biotin moiety at the 5′ end, and some primers contain inosine, which is an adenosine precursor able to form base pairs with cytidine, thymidine and adenosine (primer degeneracy).

### I.3. Reverse Hybridisation Line Probe Assay (See FIG. 4)

The VP7 or VP4 RT-PCR fragments are used to identify the HRV G genotype(s) or P genotype(s) respectively by reverse hybridisation with universal and specific probes, fixed as parallel lines onto a nitrocellulose membrane. The probes specifically hybridise to the biotinylated RT-PCR strand of the denatured PCR products from the different HRV genotypes by their interprimer region in a single-step hybridization assay. The Reverse hybridisation line probe format allows highly specific hybridization analysis under stringent conditions, permitting single mismatches between probe and target to be detected. The hybrids are detected by alkaline phosphatase conjugated streptavidin in the presence of a substrate, generating an insoluble and purple precipitating product, resulting in visible colouring of a probe line.

The VP4 Reverse hybridisation line probe assay includes 6 universal probes to confirm the amplification of VP4 sequences, and 11 specific probes to identify P4, P6, P8_{WT}, P8_{vac}, P9 and P10. The sequences are exemplified in Table 1 and Table 3. The VP7 Reverse hybridisation line probe assay includes 5 universal probes to confirm the amplification of VP7 sequences. It also includes 19 specific probes to identify G1_{WT}, G1_{vac}, G2, G3, G4, G5, G6, G8 and G9. The sequences are exemplified in Table 2 and Table 4.

### Example II

### Development of Novel Broad-Spectrum Rotavirus Primers

The objective was to design VP7 and VP4 PCR primer sets that allow PCR amplification, when needed, of a broad range of genotypes.

### II.1. VP7 Primers (G-Typing)

During the development of the assay, different primer sets have been tested, leading to the selection of those sets which were the most specific to the rotavirus gene target sequence.

The Genbank database (January-April 2004 lock point) was used to design and assess the specificity of primers.

Full-length sequences were aligned. This alignment resulted in:

1. identification of relatively conserved regions for primer design, and
2. identification of a variable interprimer region for probe design.

Based on this alignment, selection of conserved regions, suitable for design of RT-PCR primers was made to amplify VP7 sequences from at least the following 9 different HRV G-genotypes (G1, G2, G3, G4, G5, G6, G8, G9 and G12). Nucleotides are numbered according to the G1 Wa published strain GenBank accession number M21843. In this conserved region, target regions for forward and reverse primers were chosen to perform the generic VP7 RT-PCR. Six forward primers were designed, designated: ROT7For1a, 1b, 1c, 1d, 1e and 1f, targeting the 529-553 nucleic acid region. Five reverse primers were also designed, designated: ROT7Rev4a, b, c, d and e targeting the 907-928 nucleic acid region. The expected amplimer size is 400-bp comprising a 351-bp interprimer fragment. The cocktail of the 6 forward and 5 reverse primers is called the VP7 primers.

To theoretically assess the specificity of the VP7 primers, the individual sequences were compared to the corresponding HRV G-genotype reference sequences by alignment of the complete 400-bp fragments with VP7 sequences present in the GenBank database.

### Conclusions:

The sequence analyses show that, although some mismatches exist between VP7 primers and HRV G-types sequences, they are never located on a critical position of the primer such as at the 3′-end that may prevent the primer extension by the DNA polymerase activity.

### II.2. VP4 Primers (P-Typing)

The Genbank database (January-April 2004 lock point) was used to assess the specificity of primers.

Full-length sequences were aligned. This alignment resulted in:

1. identification of relatively conserved regions for primer design, and
2. identification of a variable interprimer region for probe design.

A relatively well-conserved region located between nucleotides 204 and 703 in P8 Wa published strain GenBank accession number L34161 was identified for 6 (P1, P4, P6, P8, P9 and P10) different HRV P-genotypes.

In this conserved region, target regions for forward and reverse primers were chosen to perform the generic VP4 RT-PCR. Six forward primers were designed, designated: ROT4For1a, 1b, 1c, 1d, 1e and 1f, targeting the 204-224 nucleic acid region. Three reverse primers were designed, designated: ROT4Rev1a, b, c targeting the 701-679 nucleic acid region. The expected amplimer size is 500-bp comprising a 454-bp interprimer fragment. The cocktail of the 6 forward and 3 reverse primers is called the VP4 primers.

To theoretically assess the specificity of the VP4 primers, their individual sequences were compared to the corresponding HRV P-genotype reference sequences by alignment of the complete 500-bp fragments with VP4 sequences present in the GenBank database.

### Conclusions:

Sequence analyses show that, although some mismatches exist between VP4 primers and HRV P-types sequences from the Genbank database, they are never on a critical position such as at the 3′-end of the primer.

### Example III

### Development of Novel Universal Rotavirus Probes and Type-Specific Rotavirus Probes Used for G-Typing and P-Typing

### III.1. Reverse Hybridisation Line Probe Assay Probes Used for P-Typing

The theoretical specificity of the 6 VP4 Universal and of the 11 P-specific Reverse hybridisation line probe assay probes (see Example I) was first assessed using the 2 following approaches 1) and 2) described below and further verified experimentally (section III.3 and III.4 below):
1) By comparing the ability of the Reverse hybridisation line probe assay probes to hybridize specifically with HRV of the following P-type: P4, P6, P8_{WT}, P8_{vac}, P9 and P10 found in the GenBank database. At least 1 Reverse hybridisation line probe assay Uni probe fully matches to sequences of HRV P4, P6, P8 and P9 sequences, which ensures the detection of these genotypes once the VP4 target region is amplified by the VP4 primers. The unique sequence P10 available in the Genbank database is not recognized by any of the Uni probes.

Since, based on the Genbank database, none of the Reverse hybridisation line probe assay Uniprobes fully match (theoretically) with the P10 sequence, their ability to recognize this genotype was assessed experimentally (see section III.3 and III.4 below). It was confirmed that the P10 sequence is recognized by the P10-specific probe.

Uniprobes were designed to hybridize to a spectrum of VP4 sequences, and not only to one single genotype and so allow for some mismatches in order to gain broad-spectrum detection. In other words, they are specific for HRV VP4, but do not allow recognition of single and specific types. Given that therefore the assessment of the theoretical specificity has the above limitations, when identification of a specific type needs to be done, and as some P-specific probe sequences do not fully match with HRV VP4 sequences, their ability to recognize P4, P6, P8, P9 and P10 HRV VP4 sequences target also was assessed experimentally (see section III.3 and III.4 below).
2) By comparing the probe sequences to the whole GenBank database (BLAST analysis—http://www.ncbi.nlm.nih.gov/BLAST/ performed in January 2004).

The BLAST analysis showed that 16 Reverse hybridisation line probe assay probes out of the 17 present on the VP4 strip do not recognize other HRV P-types than the one for which they have been designed. Probe lines 10 and 11 (P8_VAC-str 1b and 2b), which are specifically designed to recognize P8_{vac} HRV genotype of G1 P[8] strain deposited under ECACC 99081301), is also able to detect some P8_{WT} sequences (Finland origin).

### Conclusions:

The majority of the Reverse hybridisation line probe assay probes are specific for their corresponding P-types except one. One of the two P8_{vac} probes also recognizes a particular P8_{wt} sequence. Indeed, in that particular probe target region, both some particular WT strain and vaccine strain have the same nucleotide sequence. Therefore, although the probe itself is 100% homologous for its target, the P8_{vac} probe is not specific only for the GSK vaccine strain, in contrast to the situation in VP7, where the mutation at position 605 is in fact highly specific. This is an important difference between VP4 and VP7. In the case of a single infection by this particular P8_{wt} sequence, the interpretation of the Reverse hybridization line probe assay result will be a mixed population of P8_{wt} and P8_{vac}. However, in such a case, the G-typing will allow to assess whether or not the vaccinal strain is really present in the sample.

The theoretical specificity of the VP4/P probes was further verified experimentally (see section III.3 below).

### III.2. Reverse Hybridisation Line Probe Assay Probes Used for G-Typing

Universal probes were designed to recognize VP7 itself, whilst not being specific for a particular genotype.

Different steps were performed:

1. Identify a region of VP7 which is sufficiently conserved for general VP7 detection
2. Design of a set of universal probes, covering this region.

Universal probes have some mismatches with specific VP7 genotypes, but these are compensated by the length of the universal probes, thereby permitting general detection of VP7. The theoretical specificity of the 5 VP7 Universal and of the 19 G-specific Reverse hybridisation line probe assay probes (see Example I) was first assessed using the 2 following approaches 1) and 2) described below and further verified experimentally:

1) By comparing the Reverse hybridisation line probe assay probe sequences to known HRV G-genotype (G1_{WT}, G1_{vac}, G2, G3, G4, G5, G6, G8 and G9) sequences from the GenBank database.

Since, based on the Genbank database, none of the Reverse hybridisation line probe assay Uni probes fully matches with G3, G5, G6 and G9, their ability to recognize these genotypes by hybridization was assessed experimentally. Since some Reverse hybridisation line probe assay G-specific probes do not fully match to their VP7 target, their ability to hybridize their target sequences was assessed experimentally (see section III.3.1.3 below).

2) By comparing the Reverse hybridisation line probe assay probe sequences to the whole GenBank database (BLAST analysis—http://www.ncbi.nlm.nih.gov/BLAST/performed in January 2004)

The BLAST analysis showed that 23 Reverse hybridisation line probe assay probes out of the 24 present on the strip do not recognize other HRV G-types than the one for which they have been designed. Probe line 8 (G1_{WT}-605aC4), which is designed to recognize G1_{WT} HRV genotypes, is also able to theoretically detect some sequences, submitted to GenBank as G3 genotype.

### Conclusions:

All of the Reverse hybridisation line probe assay probes are specific for their corresponding G-types except one. One of the three G1_{WT} probe also recognizes a particular G3 sequence. Since typing using the Reverse hybridisation line probe assay is based on probe reactivities of all probes on the Reverse hybridisation line probe assay, and not on a single probe only, there was no problem for misinterpreting G1_{wt} and G3, using the complete set of probes.

The theoretical specificity of the VP7/G probes was further verified experimentally (see section III.3 below).

### III.3. Specificity of VP7 and VP4 Primers and Probes

The specificity of the primers and the probes were assessed on plasmids and on clinical material

### III.3.1. Analysis of the Specific Products Generated During VP7 and VP4 RT-PCRs on Plasmids

The specificity of the VP7 primers and of the Reverse hybridisation line probe assay G-probes used for G-typing was further evaluated using plasmids containing the VP7 region from the following HRV G-genotypes: G1_{WT}, G1_{vac}, G2, G3, G4, G5, G6, G8 and G9. The specificity of the VP4 primers and of the Reverse hybridisation line probe assay P-probes used for P-typing was further evaluated by using plasmids containing the VP4 region from the following HRV P-genotypes: P4, P6, P8_{WT}, P8_{vac}, P9 and P10.

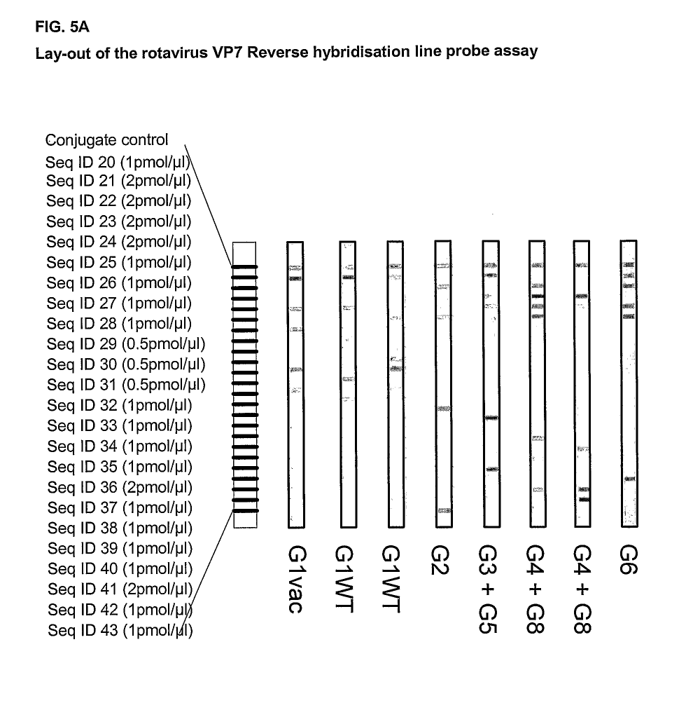

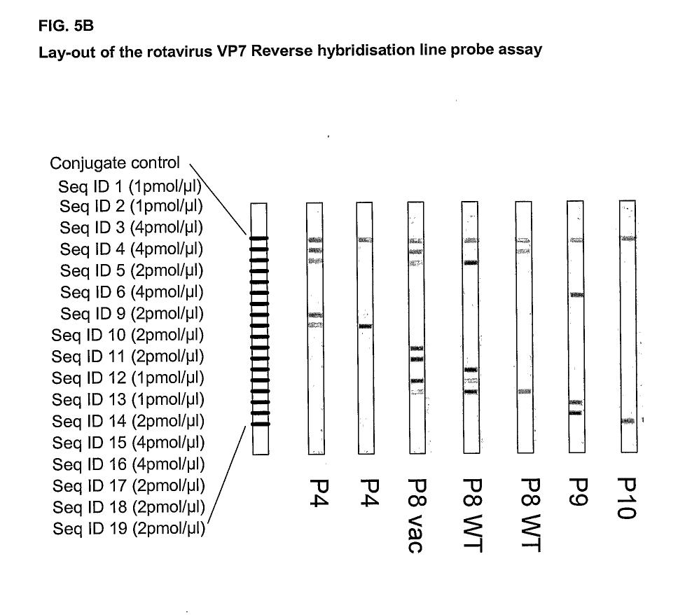

The VP7 and VP4 primers amplify a fragment of the expected size from all plasmids described above. The Reverse hybridisation line probe assay probes were able to detect all the targeted genotypes without any recognition of genotypes for which a probe was not designed. FIG. 5 gives an actual illustration of LiPA strips for the different genotypes.

III.3.2. Analysis of the specific and a specific products generated during VP7 and VP4 RT-PCRs on Clinical Samples

To assess the accuracy of the VP7 and VP4 RT-PCRs Reverse Hybridization line probe assay assays, a panel of stools was tested in parallel by Reverse hybridization line probe assay and RT-PCR includes 149 stools positive by HRV ELISA Antigen assay from either Phase II studies (95 stools) or from a panel (54 stools) received from Prof. A. D. Steele (Pretoria, South Africa). GSKBio used the Phase II algorithm based on the Gault and Gouvea RT-PCR methods for G-typing (Gouvea et al., 1990; Gault et al., 1999) and on the Khetawat RT-PCR technique for P-typing (Khetawat et al., 2001). The VP7 and VP4 RT-PCRs Reverse Hybridization line probe assay according to the algorithm.

Results are presented in Table 7 below.

TABLE 7
Nature of products generated during VP7 and VP4 RT-PCRs
on plasmids

| | G-typing | | | P-typing | | |
|---|---|---|---|---|---|---|
| Sample | RHLPA | RT-PCR | Status | RHLPA | RT-PCR | Status |
| 1 | G3 | G3 | I | P8_{WT} | P8 | I |
| 2 | G2 | G2 | I | P4 | P4 | I |
| 3 | G1_{WT} | G1_{WT} | I | P8_{WT} | P8 | I |
| 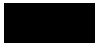 | 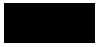 | 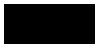 | 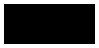 | 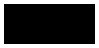 | 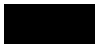 | 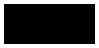 |
| 5 | G3 | G3 | I | P8_{WT} | P8 | I |
| 6 | G4 | G4 | I | P6 | P6 | I |
| 7 | G4 | G4 | I | P8_{WT} | P8 | I |
| 8 | G1_{WT} | G1_{WT} | I | P8_{WT} | P8 | I |
| 9 | G4 | G4 | I | P6 | P6 | I |
| 10 | G4 | G4 | I | P6 | P6 | I |
| 11 | G1_{WT}, | G1_{WT} | C | P8_{WT}, P6 | P6 | C |
| 12 | G3 | G3 | I | P8_{WT} | P8 | I |
| 13 | G1_{WT} | G1_{WT} | I | P8_{WT} | P8 | I |
| 14 | G1_{WT}, | G1_{WT}, | I | P4, P8_{WT} | P4 | C |
| 15 | G1_{WT}, | G1_{WT} | C | P8_{WT} | P8 | I |
| 16 | G1_{WT}, | G3 | C | P8_{WT} | P8 | I |
| 17 | G1_{WT} | G1_{WT} | I | P8_{WT} | P8 | I |
| 18 | G1_{WT} | G1_{WT} | I | P8_{WT} | P8 | I |
| 19 | G1_{WT} | G1_{WT} | I | P8_{WT} | P8 | I |
| 20 | G1_{WT} | G1_{WT} | I | P8_{WT} | P8 | I |
| 21 | G1_{WT} | G1_{WT} | I | P8_{WT} | P8 | I |
| 22 | G4 | G4 | I | P6 | P6 | I |
| 23 | G1_{WT} | G1_{WT} | I | P8_{WT} | P8 | I |
| 24 | G1_{WT} | G1_{WT} | I | P8_{WT} | P8 | I |
| 25 | G1_{WT} | G1_{WT} | I | P8_{WT}, P6 | P6 | C |
| 26 | G1_{WT} | G1_{WT} | I | P8_{WT} | P8 | I |
| 27 | G1_{WT} | G1_{WT} | I | P8_{WT} | P8 | I |
| 28 | G1_{WT} | G1_{WT} | I | P8_{WT} | P8 | I |
| 29 | G1_{WT} | G1_{WT} | I | P8_{WT} | P8 | I |
| 30 | G1_{WT} | G1_{WT} | I | P8_{WT} | P8 | I |
| 31 | G1_{WT} | G1_{WT} | I | P8_{WT} | P8 | I |
| 32 | G1_{WT} | G1_{WT} | I | P8_{WT} | P8 | I |
| 33 | G1_{WT} | G1_{WT} | I | P8_{WT} | P8 | I |
| 34 | G1_{WT} | G1_{WT} | I | P8_{WT} | P8 | I |
| 35 | G1_{WT} | G1_{WT} | I | P8_{WT} | P8 | I |
| 36 | G1_{WT} | G1_{WT} | I | P8_{WT} | P8 | I |
| 37 | G1_{WT}, | G1_{WT} | C | P8_{WT} | P8 | I |
| 38 | G9 | G9 | I | P6 | P6 | I |
| 39 | G9 | G1_{WT}, | C | P6 | P6 | I |
| 40 | G1_{WT} | G1_{WT} | I | P8_{WT}, P6 | P8 | C |
| 41 | G9 | G9 | I | P6 | P6 | I |
| 42 | G9 | G9 | I | P6 | P6 | I |
| 43 | G2, G4, | G4 | C | P4, P6, | P4 | C |
| 44 | G2 | G1_{WT}, | C | P4 | P4 | I |
| 45 | G3 | G3 | I | P8_{WT} | P8 | I |
| 46 | G2, G3 | G3 | C | P8_{WT} | P8 | I |
| 47 | G4 | G4 | I | P6 | P6 | I |
| 48 | G4 | G4 | I | P6 | P6 | I |
| 49 | G4 | G4 | I | P6 | P6 | I |
| 50 | G2, G4 | G4 | C | P6 | P6 | I |
| 51 | G3 | G3 | I | P6 | P6 | I |
| 52 | G3 | G3 | I | 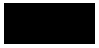 | 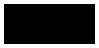 | 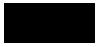 |
| 53 | G1_{WT} | G1_{WT} | I | P8_{WT} | P8 | I |
| 54 | G1_{vac} | G1_{vac} | I | 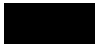 | 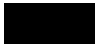 | 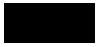 |
| 55 | G1_{vac} | G1_{vac} | I | P8_{vac} | P8 | I |
| 56 | G1_{vac} | G1_{vac} | I | P8_{vac} | P8 | I |
| 57 | G1_{vac} | G1_{vac} | I | P8_{vac} | P8 | I |
| 58 | G1_{vac} | G1_{vac} | I | 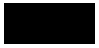 | 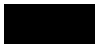 | 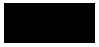 |
| 59 | G1_{WT} | G1_{WT} | I | P8_{WT} | P8 | I |
| 60 | G1_{vac} | G1_{vac} | I | P8_{vac} | P8 | I |
| 61 | G1_{WT} | G1_{WT} | I | P8_{WT} | P8 | I |
| 62 | G1_{vac} | G1_{vac} | I | P8_{vac} | P8 | I |
| 63 | G1_{vac} | G1_{vac} | I | P8_{vac} | P8 | I |
| 64 | G1_{vac} | G1_{vac} | I | P8_{vac} | P8 | I |
| 65 | G1_{vac} | G1_{vac} | I | P8_{vac} | P8 | I |
| 66 | G1_{vac} | G1_{vac} | I | P8_{vac} | P8 | I |
| 67 | G1_{WT} | G1_{WT} | I | P8_{WT} | P8 | I |
| 68 | G1_{WT} | G1_{WT} | I | P8_{WT} | P8 | I |
| 69 | 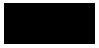 | 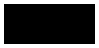 | 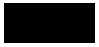 | 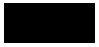 | 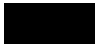 | 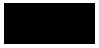 |
| 70 | G1_{WT} | G1_{WT} | I | P8_{WT} | P8 | I |
| 71 | G1_{vac} | G1_{vac} | I | P8_{vac} | P8 | I |
| 72 | G1_{vac} | G1_{vac} | I | P8_{vac} | P8 | I |
| 73 | G9 | G9 | I | P8_{WT} | P8 | I |
| 74 | G2 | G2 | I | P4 | P4 | I |
| 75 | G2 | G2 | I | P4 | P4 | I |
| 76 | G9 | G1_{WT}, | C | P8_{WT} | P8 | I |
| 77 | G2 | G2 | I | P4 | P4 | I |
| 78 | G1_{WT} | G1_{WT} | I | P8_{WT} | Neg | D |
| 79 | G1_{WT} | G1_{WT} | I | P8_{WT} | P8 | I |
| 80 | G1_{WT} | G1_{WT} | I | P8_{WT} | P8 | I |
| 81 | G1_{WT} | G1_{WT} | I | P8_{WT} | P8 | I |
| 82 | G1_{WT} | G1_{WT} | I | P8_{WT} | P8 | I |
| 83 | G1_{WT} | G1_{WT} | I | P8_{WT} | P8 | I |
| 84 | G1_{vac} | G1_{vac} | I | P8_{vac} | P8 | I |
| 85 | G1_{vac} | G1_{vac} | I | P8_{vac} | P8 | I |
| 86 | G1_{WT} | G1_{WT} | I | P8_{WT}, P4 | P8 | C |
| 87 | G1_{WT} | G1_{WT} | I | P8_{WT} | P8 | I |
| 88 | G1_{WT} | G1_{WT} | I | P8_{WT} | P8 | I |
| 89 | G1_{WT} | G1_{WT} | I | P8_{WT} | P8 | I |
| 90 | G9 | G9 | I | P8_{WT} | P8 | I |
| 91 | G1_{vac} | G1_{vac} | I | P8_{vac} | P8 | I |
| 92 | G1_{vac} | G1_{vac} | I | P8_{vac} | P8 | I |
| 93 | G1_{WT} | G1_{WT} | I | P8_{WT} | P8 | I |
| 94 | G1_{vac} | G1_{vac} | I | P8_{vac} | P8 | I |
| 95 | G1_{WT} | G1_{WT} | I | P8_{WT} | P8 | I |
| 96 | G1_{WT} | G1_{WT} | I | P8_{WT} | P8 | I |
| 97 | G1_{WT} | G1_{WT} | I | P8_{WT} | P8 | I |
| 98 | G1_{WT} | G1_{WT} | I | P8_{WT} | P8 | I |
| 99 | G1_{WT} | G1_{WT} | I | P8_{WT} | P8 | I |
| 100 | G1_{WT} | G1_{WT} | I | P8_{WT} | P8 | I |
| 101 | G1_{WT} | G1_{WT} | I | P8_{WT} | P8 | I |
| 102 | G1_{WT} | G1_{WT} | I | P8_{WT} | P8 | I |
| 103 | G1_{WT} | G1_{WT} | I | P8_{WT} | P8 | I |
| 104 | G1_{WT} | G1_{WT} | I | P8_{WT} | P8 | I |
| 105 | G1_{vac} | G1_{vac} | I | P8_{vac} | P8 | I |
| 106 | G9 | G9 | I | P6 | P6 | I |
| 107 | G2 | G2 | I | P4 | P4 | I |
| 108 | G1_{WT} | G1_{WT} | I | P8_{WT} | P8 | I |
| 109 | G9 | G9 | I | P8_{WT} | P8 | I |
| 110 | G1_{WT} | G1_{WT} | I | P8_{WT} | P8 | I |
| 111 | G1_{WT} | G1_{WT} | I | P8_{WT} | P8 | I |
| 112 | G1_{WT} | G1_{WT} | I | P8_{WT} | P8 | I |
| 113 | G9 | G9 | I | P8_{WT} | P8 | I |
| 114 | G1_{WT} | G1_{WT} | I | P8_{WT} | P8 | I |
| 115 | G1_{WT} | G1_{WT} | I | P8_{WT} | P8 | I |
| 116 | G1_{WT} | G1_{WT} | I | P8_{WT} | P8 | I |
| 117 | G1_{WT} | G1_{WT} | I | P8_{WT} | P8 | I |
| 118 | G9 | G1_{WT} | C | P8_{WT} | P8 | I |
| 119 | G1_{WT} | G1_{WT} | I | P8_{WT} | P8 | I |
| 120 | G9 | G9 | I | P8_{WT} | P8 | I |
| 121 | G1_{WT} | G1_{WT} | I | P8_{WT} | P8 | I |
| 122 | G1_{WT} | G1_{WT} | I | P8_{WT} | P8 | I |
| 123 | G9 | G9 | I | P8_{WT} | P8 | I |
| 124 | G1_{WT} | G1_{WT} | I | P8_{WT} | P8 | I |
| 125 | G1_{WT} | G1_{WT} | I | 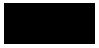 | 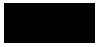 | 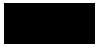 |
| 126 | G1_{WT} | G1_{WT} | I | P8_{WT} | P8 | I |
| 127 | 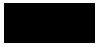 | 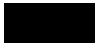 | 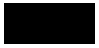 | 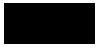 | 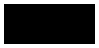 | 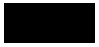 |
| 128 | G9 | G9 | I | P8_{WT} | P8 | I |
| 129 | G4 | G4 | I | P6 | P6 | I |
| 130 | G1_{WT} | G1_{WT} | I | P8_{WT} | P8 | I |
| 131 | G1_{WT}, | G1_{WT} | C | P8_{WT} | P8 | I |
| 132 | G1_{WT} | G1_{WT} | I | P8_{WT} | P8 | I |
| 133 | G1_{WT} | G1_{WT} | I | P8_{WT} | P8 | I |
| 134 | G1_{WT} | G1_{WT} | I | P8_{WT} | P8 | I |
| 135 | G1_{WT} | G1_{WT} | I | P8_{WT} | P8 | I |
| 136 | G1_{WT} | G1_{WT} | I | P8_{WT} | P8 | I |
| 137 | G1_{WT} | G1_{WT} | I | P8_{WT} | P8 | I |
| 138 | G2 | G2 | I | P4 | P4 | I |
| 139 | 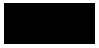 | 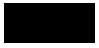 | 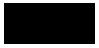 | 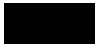 | 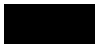 | 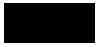 |
| 140 | G4 | G4 | I | P8_{WT} | P8 | I |
| 141 | G2 | G2 | I | P4 | P4 | I |
| 142 | G9 | G9 | I | P8_{WT} | P8 | I |
| 143 | G9 | G9 | I | P8_{WT} | P8 | I |
| 144 | G1vac | G1_{vac} | I | P8_{vac} | P8 | I |
| 145 | G9 | G9 | I | P8_{WT} | P8 | I |
| 146 | G1_{WT}, | G2 | C | P4 | P4 | I |
| 147 | G9 | G9 | I | P6 | P6 | I |
| 148 | G4 | G4 | I | P8_{WT} | P8 | I |
| 149 | G9 | G9 | I | P8_{WT} | P8 | I |

Grey zone: indicates discordant resultants between Reverse hybridization Line probe assay and Gouvea RT-PCR
RHLPA: Reverse hybridization line probe assay
I: identical
C: compatible (at least one genotype in common)
D: discordant

By comparing the results obtained by both methods, the following observations can be made:
For G-typing, 88% of the results were identical, 9% were compatible (at least one HRV genotype in common) and 3% were discordant (no HRV genotype in common). In all the cases, discordance was due to negative results obtained by using the Gault and Gouvea RT-PCR and positive results by using the Reverse Hybridization line probe assay. This seems to indicate that the Phase III testing algorithm is more sensitive as compared to the Phase II testing algorithm.
For P-typing, 90% of the results were identical, 4% were compatible (at least one HRV genotype in common) and 6% were discordant (no HRV genotype in common). Similarly to G-typing, this discordance is mainly due to negative results obtained using the Phase II testing algorithm.

### Conclusions:

The comparison between Phase II algorithm (Gault, Gouvea and Khetawat methods) and Phase III algorithm (Rota Reverse Hybridization line probe assay) shows ˜90% agreement which indicates that VP7 and VP4 RT-PCRs Reverse Hybridization line probe assays are adequate for their intended use.

This comparison showed also that the Reverse Hybridization line probe assay presents numerous advantages such as:
easier discrimination of HRV in mixed infection
higher sensitivity (mostly in P-typing)
easier archiving of the raw data (strips)
easier use as compared to other methods such as agarose gels or sequencing.
   III.3.3. Analysis of Specificity by Direct Sequencing of RT-PCR Product VP7 and/or VP4 Reverse Hybridisation Line Probe Assay Uni Probe Positive but Reverse Hybridisation Line Probe Assay Specific Probe Negative

A total of 149 samples were tested (=ELISA positive samples from PANEL 2). In some cases a positive result was observed with the Uni probe of the Reverse hybridisation line probe assay strip whereas no signal was observed with the specific probes included on the same strip. Of the 149 samples tested:
One (0.7%) was Uni VP7 probe positive but G-specific probe negative. Its sequence presents homology to a G4_{porcine} strain, as determined by DNA sequencing.
Five (3.3%) were Uni VP4 probe positive but P-specific probe negative. Their sequences were homologous to P8_{porcine} (for 4 samples) or to P6_{porcine} (for 1 sample) as determined by DNA sequencing.

### Conclusions:

All the sequenced fragments were VP7 or VP4 gene sequences. This confirms the specificity of the VP7 and VP4 primers used for the PCR amplification.
None of the sequenced Reverse hybridisation line probe assay Uni probe positive but Reverse hybridisation line probe assay specific probe negative samples corresponded to any of the genotypes for which a probe was present on the Reverse hybridisation line probe assay strip, confirming the specificity of the Reverse hybridisation line probe assay probes. All the sequenced fragments are coming from porcine strains, for which the Reverse hybridisation line probe assay does not contain specific probes. This confirms the specificity of the VP7 and VP4 typing probes to identify human rotavirus sequences.

### Example IV

### Reverse Hybridisation Line Probe Assay for the Rapid Detection and Simultaneous Identification of Several Different Rotavirus Genotypes

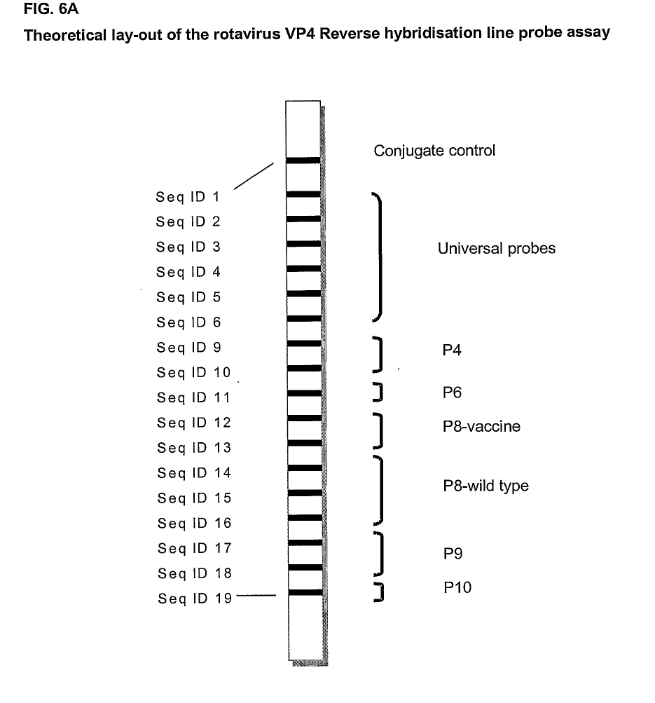

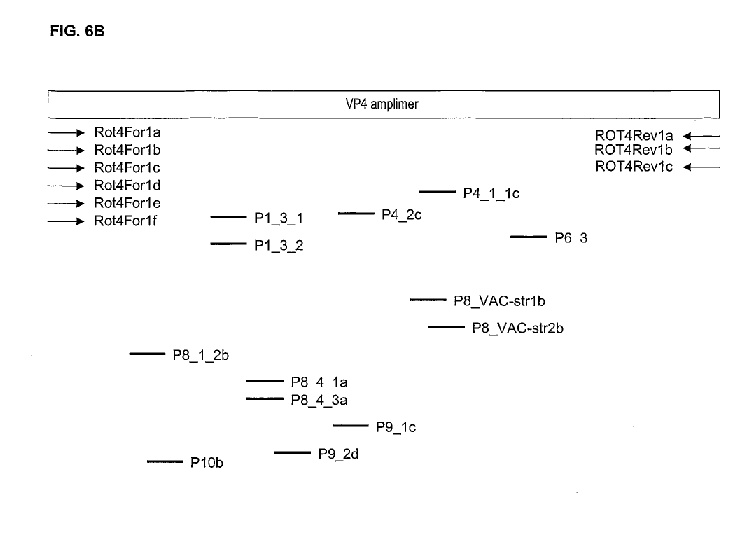

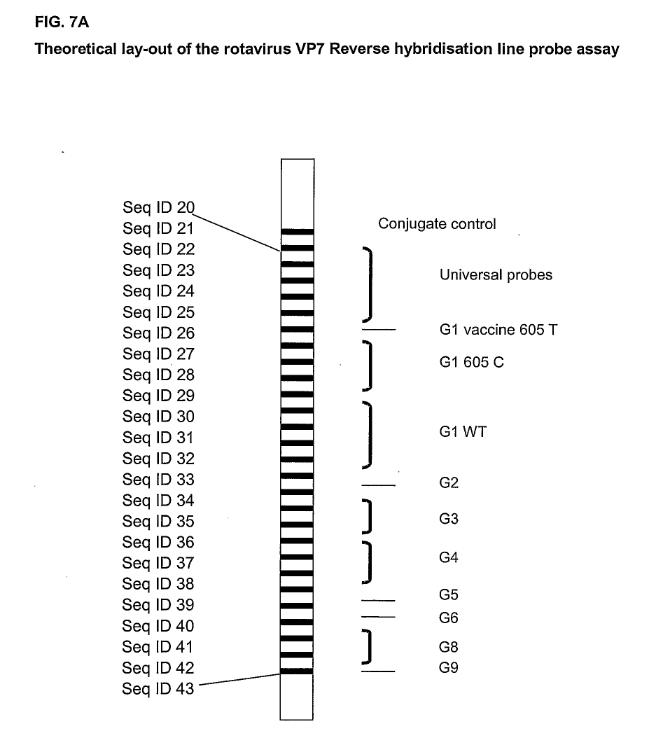

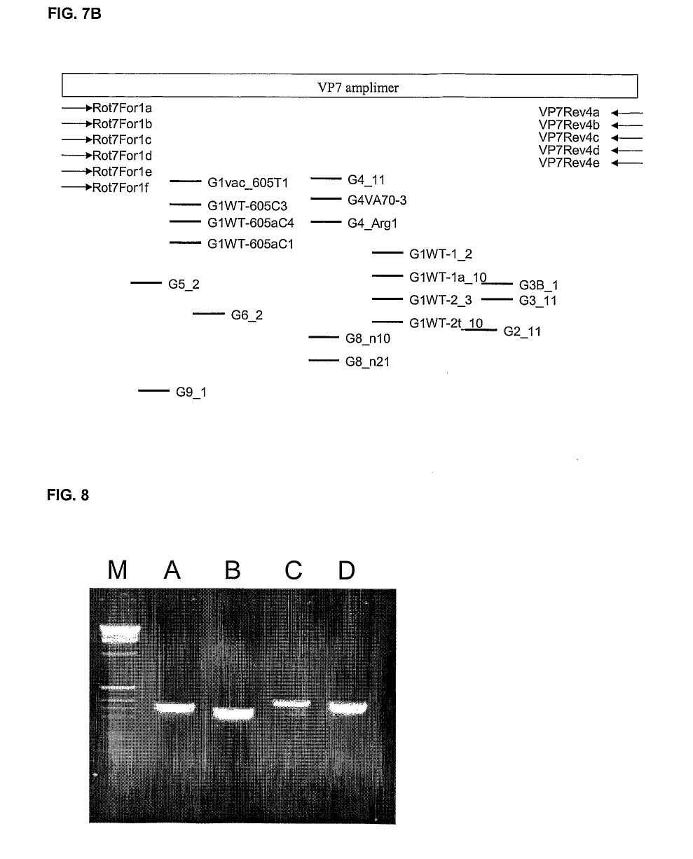

This example describes the development of two Reverse hybridisation line probe assays for the detection and identification of rotavirus genotypes. The outline of the VP4 rotavirus Reverse hybridisation line probe assay for detection and identification of P-genotypes is shown in FIG. 6A, and the outline of the VP7 rotavirus Reverse hybridisation line probe assay for the detection and identification of G-genotypes is shown in FIG. 7A.

### IV.1. Material and Methods: VP4 and VP7 Reverse Hybridisation Line Probe Assays

### IV.1.1. RNA Isolation

Total nucleic acid is extracted from the stool samples (20% w/v in EBSS) using the MagNAPure automated system and the MagNAPure LC DNA isolation kit Ill. Briefly, cells are first lysed by warming at 65° C. and addition of lysis buffer to the sample before being mixed with silica coated paramagnetic particles. These particles selectively bind nucleic acid. After separation of the paramagnetic particles from the extract, using a magnet, and several washing steps, RNA is eluted from the paramagnetic particles.

A rotavirus-positive sample is used as positive control. A dilution series is prepared, using 10-fold dilution steps. The sample, which contains the lowest concentration of rotavirus RNA and is still positive by RT-PCR (as determined by agarose gel electrophoresis) is considered as the cut-off dilution. The RNA isolation control comprises a sample containing 10 times more concentrated and is prepared in large quantities and aliquoted. 100 μl of this sample is used as positive control for each isolation run.

### IV.1.2. RT-PCR

The RNA is amplified by using RT-PCR performed with the Qiagen OneStep RT-PCR kit (QIAGEN® Ref.210212)].

For each RT-PCR run, separate positive and negative controls are included:
Positive control is made of 10 μl of the RNA, isolated from the positive control sample as mentioned above.
Negative control is made of lysis buffer and proteinase K coming from the MagNAPure kit.

### IV.1.2.1. Preparation of Multiplex RT-PCR Mix for VP4 Amplification

Prepare all 9 primers (see Table 5) at 10 pmol/μl each.

Make an equimolar mixture of these 9 primers, resulting in a final concentration of approximately 1.1 pmol/μl of each primer.

The following 6 forward primers are used: Rot4for1a, 1b, 1c, 1d, 1e, and 1f.

The following 3 reverse primers are used: Rot4rev 1a, 1b, and 1c.

Prepare the enzyme mix as follows, using components from the Qiagen One Step RT-PCR kit. As RNAse inhibitor, RNAse-out from Invitrogen is used.

TABLE 8
Preparation of multiplex RT-PCR Mix for VP4 amplification

| Component | μl/per reaction |
|---|---|
| Water | 7 |
| Q-solution (from Qiagen kit) | 10 |
| Qiagen-buffer (from Qiagen kit) | 10 |
| RNAse-out (Invitrogen) | 0.08 |
| dNTP's | 2 |
| Taq (from Qiagen kit) | 2 |
| Total volume | 31.08 |

Pipet 9 μl of the primer mix into 0.5 ml reaction tubes for each reaction. (The final amount of each primer is 10 pmoles/reaction).

Pipet 31 μl of the enzyme mix into a PCR tube.

Transfer from the clean Pre-mix PCR's lab to the extraction lab. Add 10 μl RNA-isolate to each primer mix, and mix by pipetting up and down

Incubate the RNA-primer mixes at 85° C. for 5 minutes

Place the vial containing the RNA-primer mix on ice for 2 minutes

Collect the RNA/primer mix at the bottom of the tube by brief centrifugation (˜10 sec)

Transfer the 19 μl RNA-primer mix to the PCR tubes, containing the enzyme mix.

Start the RT-PCR program immediately:

TABLE 9
RT-PCR protocol for VP4 amplification

| | |
|---|---|
| RT-step | 50° C. for 30 minutes |
| Denaturation/Taq activation | 95° C. for 15 minutes |
| 40 cycles: | 94° C. for 30 sec |
| | 52° C. for 45 sec |
| | 72° C. for 45 sec |
| Final extension | 72° C. for 10 min |

Optionally, the RT-PCR product can be analysed on an agarose gel stained by ethidium bromide.

### IV.1.2.2. Preparation of Multiplex RT-PCR Mix for VP7 Amplification

Prepare all 11 primers (see Table 6) at 10 pmol/μl each.

Make an equimolar mixture of these 11 primers, resulting in a final concentration of approximately 0.9 pmol/μl of each primer

The following 6 forward primers are used: Rot7for1a, 1b, 1c, 1d, 1e, and 1f.

The following 5 reverse primers are used: Rot7rev 4a, 4b, 4c, 4d, and 4e.

Prepare the enzyme mix as follows, using components from the Qiagen One Step RT-PCR kit. As RNAse inhibitor, RNA-out from Invitrogen is used.

TABLE 10
Preparation of multiplex RT-PCR Mix for VP7 amplification

| Component | μl/per reaction |
|---|---|
| Water | 5 |
| Q-solution (from Qiagen kit) | 10 |
| Qiagen-buffer (from Qiagen kit) | 10 |
| RNAse-out (Invitrogen) | 0.08 |
| dNTP's | 2 |
| Taq (from Qiagen kit) | 2 |
| Total volume | 29.08 |

Pipet 11 μl of the primer mix into 0.5 ml reaction tubes for each reaction (the final amount of each primer is 10 pmoles/reaction).

Pipet 29 μl of the enzyme mix into a PCR tube.

Transfer from the clean Pre-mix PCR's lab to the extraction lab.

Add 10 μl RNA-isolate to each primer mix, and mix by pipetting up and down

Incubate the RNA-primer mixes at 85° C. for 5 minutes

Place the vial containing the RNA-primer mix on ice for 2 minutes

Collect the RNA/primer mix at the bottom of the tube by brief centrifugation (˜10 sec)

Transfer the 21 μl RNA-primer mix to the PCR tubes, containing the enzyme mix.

Start the RT-PCR program immediately:

TABLE 11
RT-PCR protocol for VP7 amplification

| | |
|---|---|
| RT-step | 50° C. for 30 minutes |
| Denaturation/Taq activation | 95° C. for 15 minutes |
| 40 cycles: | 94° C. for 30 sec |
| | 55° C. for 45 sec |
| | 72° C. for 45 sec |
| Final extension | 72° C. for 10 min |

Optionally, the RT-PCR product can be analysed on an agarose gel stained by ethidium bromide.

### IV.1.3. VP4 and VP7 Reverse Hybridisation Line Probe Assay

The VP4 or VP7 RT-PCR fragments are used to identify the HRV G genotype(s) or P genotype(s) respectively by reverse hybridization with universal and specific probes, fixed as parallel lines onto a nitrocellulose membrane (see FIG. 3). The probes specifically hybridize to the biotinylated RT-PCR strand of the denatured PCR products from the different HRV genotypes by their interprimer region in a single-step hybridization assay. The Reverse hybridisation line probe assay format allows highly specific hybridization analysis under stringent conditions, permitting single mismatches between probe and target to be detected. The hybrids are detected by alkaline phosphatase conjugated streptavidin in the presence of a substrate, generating an insoluble and purple precipitating product, resulting in visible colouring of a probe line.
The VP4 Reverse hybridisation line probe assay includes 6 universal probes (Uniprobe, see Table 1) to confirm the amplification of VP4 sequences, and 11 type-specific probes (see Table 3) to identify P4, P6, P8_{WT}, P8_{vac}, P9 and P10. The lay-out of the VP4 Reverse hybridisation line probe assay is described in FIG. 6A and the probe positions are shown in FIG. 6B.
The VP7 Reverse hybridisation line probe assay includes 5 universal probes (see Table 2) to confirm the amplification of VP7 sequences. It also includes 19 type-specific probes (see Table 4) to identify G1_{WT}, G1_{vac}, G2, G3, G4, G5, G6, G8 and G9. The lay-out of the VP7 Reverse hybridisation line probe assay is described on FIG. 7A and the probe positions are shown in FIG. 7B.

For each Reverse hybridisation line probe assay run, separate positive and negative controls are performed:
The positive control corresponds to the amplified RT-PCR positive control.
The negative control corresponds to the amplified RT-PCR negative control.

The Reverse hybridisation line probe assay was performed under the following experimental conditions using the Auto-LiPA apparatus (Innogenetics, Belgium), as adapted from published procedures (Kleter et al. 1999, J. Clin. Microbiology, 37(8):2508-2517). Briefly, the following steps were performed:
Denaturation: the double-stranded PCR product (VP4 and VP7) is denatured under alkaline conditions at room temperature for 5 minutes
Hybridization: the hybridisation buffer and the reverse hybridisation line probe strip are added to the denatured material and incubated at 50° C. for 60 minutes.
Stringent washing: after 2 short washing steps (10 to 20 seconds) in the wash solution at 50° C., the strips are incubated at 50° C. in the same solution for 30 minutes.
Detection: the hybrids are detected by addition of a streptavidin-conjugate and a BCIP/NBT (5-Bromo-4-chloro-3-indolyl-phosphate/Nitroblue tetrazolium chloride) substrate

Briefly, 10 μl of the biotin labeled amplimer was mixed with 10 μl denaturation solution in a plastic trough containing the VP4 or VP7 strip. The mix was incubated for 5 minutes at room temperature. Two milliliters of pre-warmed (37° C.) hybridization buffer (3×SSC [1×SSC is 15 mM Na-citrate and 150 mM NaCl], 0.1% sodium dodecyl sulfate) was added and incubated at 50±0.5° C. for 1 h. All incubations and washing steps were performed automatically in an Auto-LiPA. The strips were washed twice for 30 s and once for 30 min at 50° C. with 2 ml of hybridization solution. Subsequently, strips were washed twice for 1 minute at room temperature with rinse solution. Following these washes, the strips were incubated with 2 ml of alkaline phosphatase-streptavidin conjugate for 30 min at room temperature. Strips were washed twice with 2 ml of rinse solution and once with 2 ml of substrate buffer. Two milliliters of substrate (5-bromo-4-chloro-3-indolylphosphate and nitroblue tetrazolium) were added and incubated for 30 min at room temperature. The reaction was stopped by washing with rinse solution for 3 and 10 minutes, respectively. A final wash is performed with 2 ml water for 10 minutes. The strips were dried and the purple colored bands were visually interpreted.

### Example V

### Evaluation of Different Primer Sets for VP7

### V.1. Objective and Outline

The objective of this experiment was to determine the efficacy of different PCR primer sets to amplify a fragment of the VP7 gene.

Four different primer sets were tested, as shown in Table 12. A cocktail of 6 forward primers (1A-1F) was used in combination with one of four reverse primers (rev1a, rev2a, rev3b, and rev4a). Each of the four reverse primers was aimed at a different conserved region of the VP7 gene.

TABLE 12
Primer sets tested for VP7 amplification

| | Reverse primer + | Amplimer | Primer |
|---|---|---|---|
| Forward primers | position¹ | size | set |
| 1A, 1B, 1C, 1D, 1E, 1F | Rev1a 907-883 | 379 bp | A |
| | tctcatcattctctcagtttgtgg | | |
| 1A, 1B, 1C, 1D, 1E, 1F | Rev2a 877-856 | 349 bp | B |
| | tcgttggatctgctgttatgtc | | |
| 1A, 1B, 1C, 1D, 1E, 1F | Rev3b 941-918 | 413 bp | C |
| | gtataaaatacttgccaccatttt | | |
| 1A, 1B, 1C, 1D, 1E, 1F | Rev4a 928-907 | 400 bp | D |
| | gccaccattttttccaattcac | | |

¹position based on G1 sequence GenBank accession M21834

The different primer combinations were tested on a serial dilution series of a VP7 G1 vaccine strain. RT-PCR was performed according to the standard protocol (shown at par IV.1.2.2.)

### V.2. Results

All 4 primer sets do generate a fragment of the expected size, as shown in FIG. 8. M=marker. Primer set C yielded the weakest band.

The four primer sets yielded the following results in the dilution series (Table 13)

TABLE 13
Amplification results

| | ~100 | ~10 | ~1 | ~0.1 | ~0.01 |
|---|---|---|---|---|---|
| Primer set | copies | copies | copy | copy | copy |
| A | + | + | − | − | − |
| B | + | + | − | − | − |
| C | − | − | − | − | − |
| D | + | + | + | − | − |

### V.3. Conclusion

Despite the fact that all 4 reverse primers were aimed at conserved target sequences in the VP7 gene, the combination of the 6 forward primers with reverse primer rev4a yielded the best results. This illustrates that the selection of primer target sequences has a significant impact on the efficacy and sensitivity of the PCR.

### Example VI

### Identification of a Novel G8 Sequence

A large series of clinical samples was routinely analysed by the VP7 PCR followed by reverse hybridization on the VP7 strip. One of the samples yielded a particular hybridization pattern on this VP7 strip, suggesting the presence of an aberrant VP7 sequence. The VP7 amplimer only hybridized to Uniprobes Uni2 and Uni4 on the VP7 strip.

This sample was further analysed by sequence analysis of the amplified VP7 sequence. A 389-basepair sequence of the VP7 amplimer was compared with all rotavirus VP7 sequences in the GenBank database, using the BLAST tool. The closest matching sequence in GenBank was AF104104.1 (strain EGY2295) with a homology score of only 86% at the nucleotide level. At the amino acid level, the sequence showed 100% homology with the translated sequence BAB18912, indicating that it was not a novel aminoacid sequence within the G8 genotype of VP7. However, at the nucleotide level, there can be significant sequence differences, which can result in aberrant genotyping results.

The G8 probe on the reverse hybridization did not recognize this G8 variant sequence, as shown in the alignment below. Therefore, an additional G8 probe was designed (see Table 4, SEQ ID NO. 70).

This example illustrates the use of the VP7 broad spectrum PCR and reverse hybridization assay as a tool to identify aberrant VP7 genotypes and variants.

We claim:

1. A method for detecting a rotaviral nucleic acid in a biological sample, comprising the steps of:
(i) contacting a nucleic acid from the sample or derived from the sample with at least one of a VP4 Uniprobe and a VP7 Uniprobe in the context of a solid support; and
(ii) detecting hybridization of the at least one uniprobe to the nucleic acid from the sample or derived from the sample;
wherein the VP4 Uniprobe is capable of hybridizing to a sequence between nucleotides 640 and 685 of a rotavirus VP4 nucleic acid, and
wherein the VP7 Uniprobe is capable of hybridizing to a sequence between nucleotides 852 and 878 of a rotavirus VP7 nucleic acid.

2. The method of claim 1, wherein the VP4 Uniprobe hybridizes to a sequence between nucleotides 640 and 685 of SEQ ID NO: 73.

3. The method of claim 1, wherein the VP7 Uniprobe hybridizes to a sequence between nucleotides 852 and 878 of SEQ ID NO: 74.

4. A method for detecting and typing a rotavirus in a biological sample, comprising the steps of:
(i) contacting at least one nucleic acid from the sample or derived from the sample with at least one VP4 type-specific probe in the context of a solid support; and
(ii) detecting type-specific hybridization between the VP4 type-specific probe and the nucleic acid in or derived from the sample;
wherein the VP4 type-specific probe is capable of hybridizing to a sequence between nucleotides 204 and 703 of a rotavirus VP4 nucleic acid.

5. The method of claim 4, wherein the VP4 type-specific probe is capable of hybridizing to a sequence between nucleotides 234 and 537 of the VP4 sequence of SEQ ID NO:73, or to a homologous sequence of another rotavirus VP4 sequence.

6. A method for detecting and typing a rotavirus in a biological sample, comprising the steps of:
(i) contacting at least one nucleic acid from the sample or derived from the sample with at least one VP7 type-specific probe in the context of a solid support; and
(ii) detecting type-specific hybridization between the VP7 type-specific probe and the nucleic acid from or derived from the sample;
wherein the VP7 type-specific probe is capable of hybridizing to a sequence between nucleotides 529 and 928 of a rotavirus VP7 nucleic acid.

7. The method of claim 4, additionally comprising the steps of:
(iii) contacting the at least one nucleic acid from the sample or derived from the sample with at least one VP7 type-specific probe in the context of a solid support; and
(iv) detecting type-specific hybridization between the VP7 type-specific probe and the nucleic acid from or derived from the sample;
wherein the VP7 type-specific probe is capable of hybridizing to a sequence between nucleotides 529 and 928 of VP7

8. The method of claim 6, wherein the VP7 type-specific probe is capable of hybridizing to a sequence between nucleotides 602 and 840 of the VP7, sequence of SEQ ID NO: 74, or to a homologous sequence of another rotavirus VP7 sequence.

9. The method of claim 6, wherein the VP4 type-specific probes are specific for at least one of: P1, P3, P4, P6, P8_{WT}, P8_{vac}, P9 and P10, and the VP7 type-specific probes are specific for at least one of G1_{WT}, G1_{vac}, G2, G3, G4, G5, G6, G8, G9 and G12.

10. The method of claim 6, wherein the VP4 type-specific probes are selected from SEQ ID NOs: 7 to 19 and 66, and wherein the VP7 type-specific probes are selected from SEQ ID NOs: 25 to 45 and 67 to 72.

11. The method of claim 6, wherein the detected nucleic acid sequences are contacted with at least one of a VP4 and a VP7 Uniprobe sequentially or simultaneously with the hybridization of step (ii).

12. The method of claim 1 or 6, further comprising the step of sequencing the detected rotavirus nucleic acid.

13. The method of claim 1 or 4, further comprising deriving at least one rotavirus nucleic acid from the sample by amplifying the at least one rotavirus nucleic acids using broad-spectrum rotavirus primers prior to step (i).

14. The method of claim 13, wherein the broad-spectrum rotavirus primers amplify at least one of a VP4 and a VP7 RNA from the sample.

15. The method of claim 14, wherein the VP4 broad-spectrum rotavirus primers comprise at least one forward (5′) primer that hybridizes to a sequence between nucleotides 204 and 224 of VP4, and at least one reverse (3′) primer that hybridizes to a sequence between nucleotides 680 and 703 of the VP4 sequence of SEQ ID NO: 73 or a homologous sequence of another rotavirus VP4 sequence.

16. The method of claim 14, wherein the VP7 broad-spectrum rotavirus primers comprise at least one forward (5′) primer that hybridizes to a sequence between nucleotides 529 and 553 of VP7, and at least one reverse (3′) primer that hybridizes to a sequence between nucleotides 907 and 928 of the VP7 sequence of SEQ ID NO: 74 or a homologous sequence of another rotavirus VP7 sequence.

17. (canceled)

18. The method of claim 15, wherein the VP4 broad-spectrum rotavirus primers are selected from SEQ ID NO: 46 to 54.

19. The method of claim 16, wherein the VP7 broad-spectrum rotavirus primers are selected from SEQ ID NO: 55 to 65.

20. A broad-spectrum rotavirus primer selected from:
a VP4 broad-spectrum rotavirus primer selected from a forward (5′) primer that hybridizes to a sequence between nucleotides 204 and 224 of VP4 and a reverse (3′) primer that hybridizes to a sequence between nucleotides 680 and 703 of the VP4 sequence of SEQ ID NO: 73 or a homologous sequence of another rotavirus; and
a VP7 broad-spectrum rotavirus primer selected from a forward (5′) primer that hybridizes to a sequence between nucleotides 529 and 553 of VP7 and a reverse (3′) primer that hybridizes to a sequence between nucleotides 907 and 928 of the VP7 sequence of SEQ ID NO: 74 or a homologous sequence of another rotavirus; and
a combination thereof.

21. A Uniprobe selected from:
a VP4 probe capable of hybridizing to a sequence between nucleotides 640 and 685 of a rotavirus VP4 nucleic acid of SEQ ID NO:73; and
a VP7 probe capable of hybridizing to a sequence between nucleotides 852 and 878 of a rotavirus VP7 nucleic acid of SEQ ID NO:74; or a combination thereof.

22. A rotavirus type-specific probe selected from:
a VP4-type specific probe capable of hybridizing to a sequence between nucleotides 204 and 703 of a rotavirus VP4 nucleic acid of SEQ ID NO:73- or to a homologous sequence of another rotavirus VP4 nucleic acid; and
a VP7-type specific probe capable of hybridizing to a sequence between nucleotides 529 and 928 of a rotavirus VP7 nucleic acid of SEQ ID NO: 74 or to a homologous sequence of another rotavirus VP7 nucleic acid.

23. A kit comprising at least two Uniprobes of claim 21.

24. A kit comprising at least two type-specific probes of claim 22.

25. The kit of claim 23 or 24, wherein the Uniprobes and type-specific probes are attached to a solid support.

26. A kit comprising at least one broad spectrum primer and at least one Uniprobe, optionally with instructions for carrying out the above methods for rotavirus detection and/or typing.

27. A kit comprising a) at least one set of VP4 Uniprobes and VP4-type specific probes, b) at least one set of VP7 Uniprobes and VP7-type specific probes, or c) a combination of both a) and b, optionally with instructions for carrying out the above methods for rotavirus detection and/or typing.

28. A kit according to claim 27, further comprising a set of broad-spectrum primers.

29. A process for detecting and typing rotavirus sequence variants in a biological sample comprising the steps of:
(i) contacting at least one of a VP7 and a VP4 Uniprobe with a nucleic acid from or derived from the biological sample;
(ii) detecting hybridization between the at least one Uniprobe and the nucleic acid from or derived from the sample; and
(iii) contacting the nucleic acid from or derived from the sample with at least one of a VP4 and a VP7 type-specific probe;
(iv) detecting hybridization between the at least one type-specific probe and the nucleic acid from or derived from the sample;
and
(iv) identifying the P- or G-type rotavirus VP4 and/or VP7 nucleic acid in the sample.

30. The method of claim 2, wherein the VP4 Uniprobe is selected from SEQ ID NOs: 1 to 6.

31. The method of claim 3, wherein the VP7 Uniprobe is selected from SEQ ID NOs: 20 to 24.


SEQUENCE LISTING (76 sequences)

SEQ ID NO: 1 - DNA, 39 bp, Artificial Sequence
  Probe sharing homoly with rotavirus nucleotide sequences
taaaccattattaatatattcattacanttagantcttg

SEQ ID NO: 2 - DNA, 36 bp, Artificial Sequence
  Probe sharing homoly with rotavirus nucleotide sequences
accgttgttaatatattcattacanttagantcttg

SEQ ID NO: 3 - DNA, 29 bp, Artificial Sequence
  Probe sharing homoly with rotavirus nucleotide sequences
ttggtggnaanccagtatttatatattca

SEQ ID NO: 4 - DNA, 30 bp, Artificial Sequence
  Probe sharing homoly with rotavirus nucleotide sequences
ttggtggtaacccnttatttatgtantcag

SEQ ID NO: 5 - DNA, 37 bp, Artificial Sequence
  Probe sharing homoly with rotavirus nucleotide sequences
ggtggtaaaccattatttatatattgtgtacacatag

SEQ ID NO: 6 - DNA, 31 bp, Artificial Sequence
  Probe sharing homoly with rotavirus nucleotide sequences
ttggaggcancccattatttatatattgcgc

SEQ ID NO: 7 - DNA, 18 bp, Artificial Sequence
  Probe sharing homoly with rotavirus nucleotide sequences
caaataggtggttagcga

SEQ ID NO: 8 - DNA, 18 bp, Artificial Sequence
  Probe sharing homoly with rotavirus nucleotide sequences
caaacagatggttagcga

SEQ ID NO: 9 - DNA, 22 bp, Artificial Sequence
  Probe sharing homoly with rotavirus nucleotide sequences
aatagacttgtaggaatgctaa

SEQ ID NO: 10 - DNA, 21 bp, Artificial Sequence
  Probe sharing homoly with rotavirus nucleotide sequences
ttcgaaatgtttaaaggtagc

SEQ ID NO: 11 - DNA, 21 bp, Artificial Sequence
  Probe sharing homoly with rotavirus nucleotide sequences
tataatagtgtttggactttc

SEQ ID NO: 12 - DNA, 20 bp, Artificial Sequence
  Probe sharing homoly with rotavirus nucleotide sequences
ctgataccagacttgtagga

SEQ ID NO: 13 - DNA, 22 bp, Artificial Sequence
  Probe sharing homoly with rotavirus nucleotide sequences
gtaggaatatttaaatatggtg

SEQ ID NO: 14 - DNA, 19 bp, Artificial Sequence
  Probe sharing homoly with rotavirus nucleotide sequences
accacctactgattactgg

SEQ ID NO: 15 - DNA, 23 bp, Artificial Sequence
  Probe sharing homoly with rotavirus nucleotide sequences
acccagtagatagacaatatatg

SEQ ID NO: 16 - DNA, 25 bp, Artificial Sequence
  Probe sharing homoly with rotavirus nucleotide sequences
gatccagtagatagacaatataatg

SEQ ID NO: 17 - DNA, 23 bp, Artificial Sequence
  Probe sharing homoly with rotavirus nucleotide sequences
acttcgtggaaatttatattatt

SEQ ID NO: 18 - DNA, 21 bp, Artificial Sequence
  Probe sharing homoly with rotavirus nucleotide sequences
agatgggcaaaatgtccaagg

SEQ ID NO: 19 - DNA, 17 bp, Artificial Sequence
  Probe sharing homoly with rotavirus nucleotide sequences
agccccattaaatgctg

SEQ ID NO: 20 - DNA, 27 bp, Artificial Sequence
  Probe sharing homoly with rotavirus nucleotide sequences
attagacataacagcagatccaacgac

SEQ ID NO: 21 - DNA, 25 bp, Artificial Sequence
  Probe sharing homoly with rotavirus nucleotide sequences
tagatatcactgctgatccaacaac

SEQ ID NO: 22 - DNA, 27 bp, Artificial Sequence
  Probe sharing homoly with rotavirus nucleotide sequences
attagatataacggctgatcccacaac

SEQ ID NO: 23 - DNA, 25 bp, Artificial Sequence
  Probe sharing homoly with rotavirus nucleotide sequences
tagatattacagctgatccgacgac

SEQ ID NO: 24 - DNA, 26 bp, Artificial Sequence
  Probe sharing homoly with rotavirus nucleotide sequences
ttggatattacngcngatccaacaac

SEQ ID NO: 25 - DNA, 18 bp, Artificial Sequence
  Probe sharing homoly with rotavirus nucleotide sequences
gaatacgcaaatgttagg

SEQ ID NO: 26 - DNA, 18 bp, Artificial Sequence
  Probe sharing homoly with rotavirus nucleotide sequences
aatacgcaaacgttagga

SEQ ID NO: 27 - DNA, 20 bp, Artificial Sequence
  Probe sharing homoly with rotavirus nucleotide sequences
actgaatacacaaacgttag

SEQ ID NO: 28 - DNA, 20 bp, Artificial Sequence
  Probe sharing homoly with rotavirus nucleotide sequences
ctgaatatacaaacgttagg

SEQ ID NO: 29 - DNA, 21 bp, Artificial Sequence
  Probe sharing homoly with rotavirus nucleotide sequences
ttagctatagtggatgtcggg

SEQ ID NO: 30 - DNA, 24 bp, Artificial Sequence
  Probe sharing homoly with rotavirus nucleotide sequences
aaattagctatagtagatgtcggg

SEQ ID NO: 31 - DNA, 19 bp, Artificial Sequence
  Probe sharing homoly with rotavirus nucleotide sequences
cgttgatgggataaatcat

SEQ ID NO: 32 - DNA, 19 bp, Artificial Sequence
  Probe sharing homoly with rotavirus nucleotide sequences
cgttgatgggataaattat

SEQ ID NO: 33 - DNA, 21 bp, Artificial Sequence
  Probe sharing homoly with rotavirus nucleotide sequences
agaaaatgttgctataattca

SEQ ID NO: 34 - DNA, 19 bp, Artificial Sequence
  Probe sharing homoly with rotavirus nucleotide sequences
agcagttatacaggttggt

SEQ ID NO: 35 - DNA, 16 bp, Artificial Sequence
  Probe sharing homoly with rotavirus nucleotide sequences
ggcggtcatacaagtt

SEQ ID NO: 36 - DNA, 20 bp, Artificial Sequence
  Probe sharing homoly with rotavirus nucleotide sequences
cagctacttttgaaacagtt

SEQ ID NO: 37 - DNA, 19 bp, Artificial Sequence
  Probe sharing homoly with rotavirus nucleotide sequences
agctacttttgaaatggtg

SEQ ID NO: 38 - DNA, 20 bp, Artificial Sequence
  Probe sharing homoly with rotavirus nucleotide sequences
cagctacttttgaaacgggg

SEQ ID NO: 39 - DNA, 20 bp, Artificial Sequence
  Probe sharing homoly with rotavirus nucleotide sequences
tatctatgggttcttcatgg

SEQ ID NO: 40 - DNA, 17 bp, Artificial Sequence
  Probe sharing homoly with rotavirus nucleotide sequences
ctcggtatcggatgtct

SEQ ID NO: 41 - DNA, 23 bp, Artificial Sequence
  Probe sharing homoly with rotavirus nucleotide sequences
actacaacttttgaagaagttgc

SEQ ID NO: 42 - DNA, 20 bp, Artificial Sequence
  Probe sharing homoly with rotavirus nucleotide sequences
acactacgacttttgaagaa

SEQ ID NO: 43 - DNA, 17 bp, Artificial Sequence
  Probe sharing homoly with rotavirus nucleotide sequences
atgggacagtcttgtac

SEQ ID NO: 44 - DNA, 16 bp, Artificial Sequence
  Probe sharing homoly with rotavirus nucleotide sequences
gaagaggtagcaaacg

SEQ ID NO: 45 - DNA, 15 bp, Artificial Sequence
  Probe sharing homoly with rotavirus nucleotide sequences
aggtagcaaacgcgg

SEQ ID NO: 46 - DNA, 21 bp, Artificial Sequence
  Primer sharing homoly with rotavirus nucleotide sequences
agatggtccttatcagccaac

SEQ ID NO: 47 - DNA, 20 bp, Artificial Sequence
  Primer sharing homoly with rotavirus nucleotide sequences
ganggtccttatcagcctac

SEQ ID NO: 48 - DNA, 21 bp, Artificial Sequence
  Primer sharing homoly with rotavirus nucleotide sequences
agatggtccttatcaacctac

SEQ ID NO: 49 - DNA, 21 bp, Artificial Sequence
  Primer sharing homoly with rotavirus nucleotide sequences
cgatggtccttatcaaccaac

SEQ ID NO: 50 - DNA, 20 bp, Artificial Sequence
  Primer sharing homoly with rotavirus nucleotide sequences
gacggaccatatcaaccgac

SEQ ID NO: 51 - DNA, 20 bp, Artificial Sequence
  Primer sharing homoly with rotavirus nucleotide sequences
gatggtccntatcaaccnac

SEQ ID NO: 52 - DNA, 22 bp, Artificial Sequence
  Primer sharing homoly with rotavirus nucleotide sequences
ttncttgtattctgnattggtg

SEQ ID NO: 53 - DNA, 24 bp, Artificial Sequence
  Primer sharing homoly with rotavirus nucleotide sequences
catttctagtattttgaattggtg

SEQ ID NO: 54 - DNA, 23 bp, Artificial Sequence
  Primer sharing homoly with rotavirus nucleotide sequences
catttctagtgttttgtatcggt

SEQ ID NO: 55 - DNA, 25 bp, Artificial Sequence
  Primer sharing homoly with rotavirus nucleotide sequences
aatgaatggttatgtaatccaatgg

SEQ ID NO: 56 - DNA, 25 bp, Artificial Sequence
  Primer sharing homoly with rotavirus nucleotide sequences
aangaatggctgtgcaatccnatgg

SEQ ID NO: 57 - DNA, 25 bp, Artificial Sequence
  Primer sharing homoly with rotavirus nucleotide sequences
aangagtggttatgtaatccnatgg

SEQ ID NO: 58 - DNA, 25 bp, Artificial Sequence
  Primer sharing homoly with rotavirus nucleotide sequences
aatgaatggttatgnaacccaatgg

SEQ ID NO: 59 - DNA, 25 bp, Artificial Sequence
  Primer sharing homoly with rotavirus nucleotide sequences
aatgagtggctttgtaatccaatgg

SEQ ID NO: 60 - DNA, 25 bp, Artificial Sequence
  Primer sharing homoly with rotavirus nucleotide sequences
aatcaatggttatgtaatccgatgg

SEQ ID NO: 61 - DNA, 22 bp, Artificial Sequence
  Primer sharing homoly with rotavirus nucleotide sequences
gccaccattttttccaattcac

SEQ ID NO: 62 - DNA, 22 bp, Artificial Sequence
  Primer sharing homoly with rotavirus nucleotide sequences
gccaccattttttccaatttat

SEQ ID NO: 63 - DNA, 22 bp, Artificial Sequence
  Primer sharing homoly with rotavirus nucleotide sequences
gccaccattttttccaatttac

SEQ ID NO: 64 - DNA, 22 bp, Artificial Sequence
  Primer sharing homoly with rotavirus nucleotide sequences
gccaccatttcttccaattaac

SEQ ID NO: 65 - DNA, 22 bp, Artificial Sequence
  Primer sharing homoly with rotavirus nucleotide sequences
gccaccattttttccagttcac

SEQ ID NO: 66 - DNA, 25 bp, Artificial Sequence
  Priobe sharing homoly with rotavirus nucleotide sequences
agtccagtggatagacaatatactg

SEQ ID NO: 67 - DNA, 21 bp, Artificial Sequence
  Probe sharing homoly with rotavirus nucleotide sequences
agagaatgttgctataattca

SEQ ID NO: 68 - DNA, 19 bp, Artificial Sequence
  Probe sharing homoly with rotavirus nucleotide sequences
agcagttatacaggtaggt

SEQ ID NO: 69 - DNA, 20 bp, Artificial Sequence
  Probe sharing homoly with rotavirus nucleotide sequences
cagttacttttgaaacagtt

SEQ ID NO: 70 - DNA, 23 bp, Artificial Sequence
  Probe sharing homoly with rotavirus nucleotide sequences
acgaccactttcgaggaagttgc

SEQ ID NO: 71 - DNA, 18 bp, Artificial Sequence
  Probe sharing homoly with rotavirus nucleotide sequences
atgggacaatcttgtacc

SEQ ID NO: 72 - DNA, 18 bp, Artificial Sequence
  Probe sharing homoly with rotavirus nucleotide sequences
ataggacaatcttgtacc

SEQ ID NO: 73 - DNA, 2359 bp, Rotavirus
ggctataaaatggcttcactcatttatagacagcttctcactaattcatattcagtagatttacatgatgaaatagagcaaattggatcagaaaaaactcaaaatgtaactataaatccgagtccatttgcacagactagatatgctccagtcaattggggtcatggagagataaatgattcgactacagtagaaccaatgttagatggtccttatcagccaactacatttactccacctaatgattattggatacttattaattcaaatacaaatggagtagtatatgaaagtacaaataatagtgacttttggactgcagtcgttgctattgaaccgcacgttaacccagtagatagacaatatacgatatttggtgaaagtaagcaatttaatgtgagtaacgattcaaataaatggaagtttttagaaatgtttagaagcagtagtcaaaatgaattttataatagacgtacattaacttctgatactagatttgtaggaatattgaaatatggtggaagagtatggacatttcatggtgaaacaccaagagctactactgacagttcaagtactgcaaatttaaataatatatcaattacaattcattcagaattttacattattccaaggtcccaagaatctaaatgtaatgaatatattaataatggtctgccaccaattcaaaatactagaaatgtagttccattgccattatcatctagatcgatacagtataagagagcacaagttaatgaagacattatagtttcaaaaacttcattatggaaagaaatgcagtataatagggatattataattagatttaaatttggtaatagtattgtaaaaatgggaggactaggttataaatggtctgaaatatcatataaggcagcaaattatcaatataattacttacgtgacggtgaacaagtaaccgcacacaccacttgttcagtaaatggagtgaacaattttagctataatggagggtttctacccactgattttggtatttcaaggtatgaagttattaaagagaattcctatgtatatgtagactattgggatgattcaaaagcatttagaaatatggtatatgttagatcattagcagctaatttgaattcagtgaaatgtacaggtggaagttataattttagtataccagtaggtgcatggccagtaatgaatggtggcgctgtttcgttgcattttgccggagttacattatccacgcaatttactgattttgtatcattgaattcactacgatttagatttagtttgacagttgatgaaccacctttctcaatattgagaacacgtacagtgaatttgtacggattaccagccgctaatccaaataatggaaatgaatactacgaaatatcaggaaggttttcactcatttatttagttccaactaatgatgattatcagactccaattatgaattcagtgacagtaagacaagatttagagcgccaacttactgatttgcgagaagaatttaactcattgtcacaagaaatagctatggcacaattgattgatttagcactgttgcctctagatatgttttccatgttttcaggaattaaaagtacaattgatttaactaaatcaatggcgactagtgtaatgaagaaatttagaaaatcaaaattagctacatcaatttcagaaatgactaattcattgtcagatgctgcttcatcagcatcaagaaacgtttctattagatcgaatttatctgcgatctcaaattggactaatgtttcaaatgatgtgtcaaacgtaactaattcattgaacgatatttcaacacaaacgtctacaattagtaagaaatttagattaaaagaaatgattactcaaactgaaggaatgagctttgacgacatttcagcagctgtactaaaaacaaaaatagatatgtctactcaaattggaaaaaatactttacctgacatagttacagaggcatctgagaaatttattccaaaacgatcatatcgaatattaaaggatgatgaagtaatggaaattaatactgaaggaaaattctttgcatacaaaattaatacatttgatgaagtgccattcgatgtaaataaattcgctgaactagtaacagattctccagttatatcagcgataatcgattttaagacattgaaaaatttaaatgataattatggaatcactcgtacagaagcgttaaatttaattaaatcgaatccaaatatgttgcgtaatttcattaatcaaaataatccaattataaggaatagaattgaacagttaatactacaatgtaaattgtgagaacgctattgaggatgtgacc

SEQ ID NO: 74 - DNA, 1059 bp, Rotavirus
ggctttaaaagagagaatttccgtctggctaacggttagctccttttaatgtatggtattgaatataccacaattctaatctttttgatatcaatcattctactcaactatatattaaaatcagtgactcgaataatggactacattatatatagatttttgttgattactgtagcattatttgctttgacaagagctcagaattatggacttaacttaccaataacaggatcaatggacgctgtatatactaactctactcaagaagaagtgtttctaacttctacgttatgtctgtattatccaactgaagcaagtactcaaatcaatgatggtgactggaaagactcattgtcgcaaatgtttcttacaaagggttggccaacaggatctgtttactttaaagagtactcaaatattgttgatttttctgttgacccacagctgtattgtgactataatttagtacttatgaaatatgaccaaagtcttaaattagatatgtcagagttagctgatttaatattgaatgaatggttatgtaacccaatggatgtaacattatactattatcaacaatcgggagaatcaaataagtggatatcgatgggatcatcatgtaccgtgaaagtgtgtccgctaaatacacaaacgttagggataggttgtcaaacaacaaacgtagactcatttgaaatgattgctgagaatgagaaattagctatagtggatgtcgttgatgggataaatcataaaataaatttaacaactacgacatgtactattcgaaattgtaagaaattaggtccaagagaaaatgtagctgtaatacaagttggtggttctaatgtgttagacataacagcagatccaacaactaatccacaaactgagagaatgatgagagtgaattggaaaaagtggtggcaagtattttatactatagtagattatattaatcaaattgtacaggtaatgtccaaaagatcaagatcattaaattctgcagctttttattatagagtatagtatcttagattagaattgttcgatgtgacc

SEQ ID NO: 75 - DNA, 2350 bp, Rotavirus
atggcttcactcatttatagacaacttctcactaattcatattcagtagatttacatgatgaaatagagcaaattggatcagaaaaaactcagaatgtaactataaatccgggtccatttgcacagactagatatgctccagtcaattgggatcatggagagataaatgattcgactacagtagaaccaattttagatggtccttatcagccaactacatttactccacctaatgattattggatacttattaattcaaatacaaatggagtagtatatgaaagtacaaataatagtgacttttggactgcagtcgttgctattgaaccgcacgtcaacccagtagatagacaatatatgatatttggtgaaagcaagcaatttaatgtgagtaacgattcaaataaatggaagtttttagaaatgtttagaagcagtagtcaaaatgaattttataatagacgtacattaacttctgataccagacttgtaggaatatttaaatatggtggaagagtatggacatttcatggtgaaacaccgagagctactactgacagttcaagtactgcaaatttaaataatatatcaattacaattcattcagaattttacattattccaaggtcccaggaatctaaatgtaatgaatatattaataatggtctgccaccaattcaaaatactagaaatgtagttccattgccattatcatctagatcgatacagtataagagagcacaagttaatgaagacattatagtttcaaaaacttcattatggaaagaaatgcagtataatagggatattataattagatttaaatttggtaatagtattgtaaagatgggaggactaggttataaatggtctgaaatatcatataaggcagcaaattatcaatataattacttacgtgacggtgaacaagtaaccgcacacaccacttgttcagtaaatggagtgaacaattttagctataatggagggtttctacccactgattttggtatttcaaggtatgaagttattaaagagaattcttatgtatatgtagactattgggatgattcaaaagcatttagaaatatggtatatgttagatcattagcagctaatttaaattcagtgaaatgtacaggtggaagttattatttcagtataccagtaggtgcatggccagtaatgaatggtggcgctgtttcgttgcattttgccggagttacattatccacgcaatttactgattttgtatcattaaattcactacgatttagatttagtttgacagttgatgaaccacctttctcaatactgagaacacgtacagtgaatttgtatggattaccagccgctaatccaaataatggaaatgaatactacgaaatatcaggaaggttttcactcatttctttagttccaactaatgatgattatcagactccaattatgaattcagtgacggtaagacaagatttagagcgccaacttactgatttacgagaagaatttaactcattgtcacaagaaatagctatggcacaattgattgatttagcactgttgcctctagatatgttttccatgttttcaggaattaaaagtacaattgatttaactaaatcaatggcgactagtgtaatgaagaaatttagaaaatcaaaattagctacatcaatttcagaaatgactaattcattgtcagatgctgcttcatcagcatcaagaaacgtttctattagatcgaatttatctgcgatttcaaattggactaatgtttcaaatgatgtgtcaaacgtaactaattcattgaacgatatttcaacacaaacatctacaattagtaagaaacttagattaaaagaaatgattactcaaactgaaggaatgagctttgacgacatttcagcagctgtactaaaaacaaaaatagatatgtctactcaaattggaaaaaatactttacctgatatagttacagaagcatctgagaaatttattccaaaacgatcatatcgaatattaaaggatgatgaagtaatggaaattaatactgaaggaaaattctttgcatacaaaattaatacatttgatgaagtgccattcgatgtaaataaattcgctgaactagtaacagattctccagttatatcagcgataatcgattttaagacattgaaaaatttaaatgataattatggaatcactcgtacagaagcgttaaatttaattaaatcgaatccaaatatgttacgtaatttcattaatcaaaataatccaattataaggaatagaattgaacagttaatactacaatgtaaattgtgagaacgctattgaggatgtgacc

SEQ ID NO: 76 - DNA, 1009 bp, Rotavirus
atgtatggtcttgaatataccacaattctaatctttctgatatcaattattctactcaactatatattaaaatcagtaactcgaataatggactacattatatatagatctttgttgatttatgtagcattatttgccttgacaagagctcagaattatgggcttaacttaccaataacaggatcaatggacactgtatacgctaactctactcaagaaggaatatttctaacatccacattatgtttgtattatccaactgaagcaagtactcaaattaatgatggtgaatggaaagactcattgtcacaaatgtttctcacaaaaggttggccaacaggatcagtctattttaaagagtattcaagtattgttgatttttctgtcgatccacaattatattgtgattataacttagtactaatgaaatatgatcaaaatcttgaattagatatgtcagagttagctgatttaatattgaatgaatggttatgtaatccaatggatataacattatattattatcaacaatcgggagaatcaaataagtggatatcaatgggatcatcatgtactgtgaaagtgtgtccactgaatacgcaaatgttaggaataggttgtcaaacaacaaatgtagactcgtttgaaatggttgctgagaatgagaaattagctatagtggatgtcgttgatgggataaatcataaaataaatttgacaactacgacatgtactattcgaaattgtaagaagttaggtccaagagagaatgtagctgtaatacaagttggtggctctaatgtattagacataacagcagatccaacgactaatccacaaactgagagaatgatgagagtgaattggaaaaaatggtggcaagtattttatactatagtagattatattaaccaaatcgtgcaggtaatgtccaaaagatcaagatcattaaattctgcagctttttattatagagtatagatatatcttagattagatcgatgtgacc